(19) United States
(12) Patent Application Publication
(10) Pub. No.: US 2024/0190845 A1
(43) Pub. Date: Jun. 13, 2024

(54) CRYSTAL FORMS OF 6-(CYCLOPROPANECARBOXAMIDO)-4-((2-METHOXY-3-(1-METHYL-1H-1,2,4-TRIAZOL-3-YL)PHENYL)AMINO)-N-(METHYL-D3)PYRIDAZINE-3-CARBOXAMIDE

(71) Applicant: Bristol-Myers Squibb Company, Princeton, NJ (US)

(72) Inventors: Candice Y. Choi, Fort Lee, NJ (US); Daniel Richard Roberts, Robbinsville, NJ (US); Chenkou Wei, Princeton Junction, NJ (US); Marta Dabros, Foster City, CA (US); Franz Lembke, Bend, OR (US); Ian Yates, Bend, OR (US); Natalie Louise Kelk, Edinburgh (GB); David James Pearson, Lauder (GB)

(21) Appl. No.: 18/284,531

(22) PCT Filed: Mar. 24, 2022
(86) PCT No.: PCT/US2022/021815

§ 371 Date: Sep. 27, 2023

Related U.S. Application Data
(60) Provisional application No. 63/167,504, filed on Mar. 29, 2021.

(51) Int. Cl. C07D 403/12

(52) U.S. Cl. CPC C07D 403/12

(57) ABSTRACT

Provided are several crystalline forms of 6-(cyclopropanecarboxamido)-4-((2-methoxy-3-(1-methyl-1H-1,2,4 -triazol-3-yl)phenyl) amino)-N-(methyl-d₃)pyridazine-3-carboxamide: Form F, Form G, Form H, Form I, Form J, and Form K. Certain of these crystalline forms are hydrates. Characterization data for each of these crystalline forms are provided.

### FIELD OF THE INVENTION

The present invention generally relates to crystalline forms of 6-(cyclopropanecarboxamido)-4-((2-methoxy-3-(1-methyl-1H-1,2,4-triazol-3-yl)phenyl) amino)-N-(methyl-d₃)pyridazine-3-carboxamide. Such crystalline forms include crystalline hydrate forms of 6-(cyclopropanecarboxamido)-4-((2-methoxy-3-(1-methyl-1H-1,2,4-triazol-3-yl)phenyl) amino)-N-(methyl-d₃)pyridazine-3-carboxamide, as well as a crystalline anhydrous form of 6-(cyclopropanecarboxamido)-4-((2-methoxy-3-(1-methyl-1H-1,2,4-triazol-3-yl)phenyl) amino)-N-(methyl-d₃)pyridazine-3-carboxamide. The present invention also generally relates to pharmaceutical compositions comprising the crystalline forms, as well methods for obtaining such crystalline forms.

### BACKGROUND OF THE INVENTION

The compound, 6-(cyclopropanecarboxamido)-4-((2-methoxy-3-(1-methyl -1H-1,2,4-triazol-3-yl)phenyl)amino)-N-(methyl-d₃)pyridazine-3-carboxamide, has the structure of Formula (I):

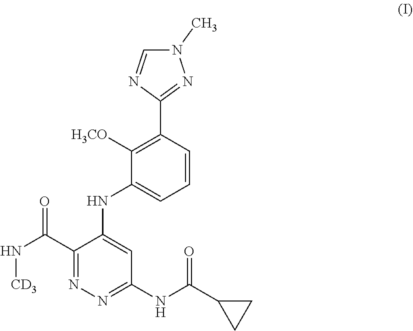 [SMILES: [2H]C([2H])([2H])NC(=O)c1nnc(NC(=O)C2CC2)cc1Nc1cccc(-c2ncn(C)n2)c1OC]

and is referred to herein as “Compound (I)”. Compound (I) is disclosed in U.S. Pat. No. RE47,929 E, which is assigned to the present assignee. U.S. Pat. No. RE47,929 E also discloses methods of treatment employing Compound (I). Compound (I) is also known as Deucravacitinib.

Compound (I) is a Tyk2 inhibitor currently in clinical trials for the treatment of autoimmune and auto-inflammatory diseases such as psoriasis, psoriatic arthritis, lupus, lupus nephritis, Sjögren's syndrome, inflammatory bowel disease, Crohn's disease, and ankylosing spondylitis.

In the synthesis of a chemical compound intended for pharmaceutical use, it is necessary to isolate and purify the compound at the completion of the synthesis process and prior to further processing to provide the compound in a pharmaceutical formulation. The isolation and the purification steps, which can be combined or separate consecutive steps, provide the compound as a purified solid, ideally with minimal loss of yield during isolation of the compound from other components of the reaction mixture and/or during purification to remove impurities from the isolated compound sample.

It is desirable to provide a solid form of such a compound that can be reproducibly produced from the isolation and/or purification steps.

Further, it is desirable to isolate the compound in a solid form that is physically and chemically stable upon storage, including at different conditions of temperature and humidity. It is also desirable to provide a compound in a solid form that is amenable to additional processing, for example a crystalline form that can be converted to other solid forms, such as an amorphous form or other crystalline forms.

As described herein, crystalline forms of Compound (I) are surprisingly amenable to additional processing and can be converted to other solid forms. The present invention is directed to these and other important aspects.

### SUMMARY OF THE INVENTION

The present invention provides crystalline forms Compound (I), namely Form F, Form G, Form H, Form I, Form J, and Form K. The name used herein to characterize a specific form, e.g. “Form F”, “Form G”, etc. should not be considered limiting with respect to any other substance possessing similar or identical physical and chemical characteristics, but rather it should be understood that this designation is a mere identifier that should be interpreted according to the characterization information also presented herein.

### BRIEF DESCRIPTION OF THE DRAWINGS

FIG. 1 shows an observed powdered X-ray diffraction pattern (CuKα, measured at room temperature) of crystalline Form F of Compound (I).

FIG. 2 shows an observed powdered X-ray diffraction pattern (CuKα, measured at room temperature) of crystalline Form G of Compound (I).

FIG. 3 shows an observed powdered X-ray diffraction pattern (CuKα, measured at room temperature) of crystalline Form H of Compound (I).

FIG. 4 shows an observed powdered X-ray diffraction pattern (CuKα, measured at room temperature) of crystalline Form I of Compound (I).

FIG. 5 shows an observed powdered X-ray diffraction pattern (CuKα, measured at room temperature) of crystalline Form J of Compound (I).

FIG. 6 shows a differential scanning calorimetry (DSC) thermogram of crystalline Form J of Compound (I).

FIG. 7 shows a thermogravimetric analysis (TGA) thermogram of Form J of Compound (I).

FIG. 8 shows an observed powdered X-ray diffraction pattern (CuKα, measured at room temperature) of crystalline Form K of Compound (I).

FIG. 9 shows a differential scanning calorimetry (DSC) thermogram of crystalline Form K of Compound (I).

FIG. 10 shows a thermogravimetric analysis (TGA) thermogram of Form K of Compound (I).

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

The names used herein to characterize a specific form, e.g., “Form F” etc., are merely identifiers that are to be interpreted in accordance with the characterization information presented herein (or in references cited herein) and are not to be limited so as to exclude any other substance possessing similar or identical physical and chemical characteristics.

The definitions set forth herein take precedence over definitions set forth in any patent, patent application, and/or patent application publication incorporated herein by reference.

All numbers expressing quantities of ingredients, weight percentages, temperatures, and so forth that are preceded by the word “about” are to be understood as only approximations so that slight variations above and below the stated number may be used to achieve substantially the same results as the stated number. Accordingly, unless indicated to the contrary, numerical parameters preceded by the word “about” are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

All measurements are subject to experimental error, consistent with the spirit of the invention.

As used herein, “polymorphs” refer to crystalline forms having the same chemical structure but different spatial arrangements of the molecules and/or ions forming the crystals.

As used herein, “hydrate” refers to having a stoichiometric or non-stoichiometric amount of water molecules incorporated into the crystalline lattice structure. In some embodiments, a stoichiometric amount may be specified (e.g., monohydrate).

As used herein, “amorphous” refers to a solid form of a molecule and/or ion that is not crystalline. An amorphous solid does not display a definitive X-ray diffraction pattern with sharp maxima.

As used herein, “substantially pure,” when used in reference to a crystalline form of a compound, means having the crystalline form at a purity greater than 90 weight %, including greater than 90, 91, 92, 93, 94, 95, 96, 97, 98, and 99 weight %, and also including equal to about 100 weight %, based on the weight of the sample or specimen. The remaining material comprises other form(s) of the compound, and/or reaction impurities and/or processing impurities arising from its preparation. For example a crystalline form of Compound (I) may be deemed substantially pure in that it has a purity greater than 90 weight %, as measured by means that are at this time known and generally accepted in the art, where the remaining less than 10 weight % of material comprises amorphous and/or other form(s) of Compound (I) and/or reaction impurities and/or processing impurities.

As used herein, a powder X-ray diffraction (PXRD) pattern “comprising” a number of peaks selected from a specified group of peaks, is intended to include PXRD patterns having additional peaks that are not included in the specified group of peaks. For example, a PXRD pattern comprising four or more (or five or more, etc.) peaks or 2θ values selected from: a, b, c, d, e, f, g, and h, is intended to include a PXRD pattern having: (a) four or more (or five or more, etc.) 2θ values selected from: a, b, c, d, e, f, g, and h; and (b) zero, one, or more peaks that are not one of peaks a, b, c, d, e, f, g, and h.

The presence of reaction impurities and/or processing impurities may be determined by analytical techniques known in the art, such as, for example, chromatography, nuclear magnetic resonance spectroscopy, mass spectrometry, and/or infrared spectroscopy.

As used herein, the unit cell parameter “molecules per unit cell” refers to the number of molecules of Compound (I) in the unit cell.

Forms of Compound (I) have been described in WO 2018/183656 (Form A), WO 2019/232138 (Form B), WO 2020/251911 (Forms C and D), and U.S. Provisional Patent Application No. 63/143,769 (Form E). The present invention generally relates to Form F, Form G, Form H, Form I, Form J, and Form K of Compound (I).

### Form F of Compound (I)

In some embodiments, Compound (I) is provided as a crystalline material comprising Form F. The crystalline Form F of Compound (I) is a free base hydrate crystalline form.

In certain embodiments, crystalline Form F of Compound (I) is characterized by unit cell parameters approximately equal to the following:
Cell dimensions (monoclinic crystal system):
   a=54.50±0.10 ∪
   b=7.83±0.10 ∪
   c=23.43±0.10 ∪
   α=90.0°
   β=102.4±1.0°
   γ=90.0°
Space group: C2/c
Molecules per unit cell (Z): 16
Unit cell volume=9766±20 Å³
Density (calculated)=1.288 mg/m³,
   wherein the unit cell parameters of Form F of Compound (I) are measured at a temperature of about 296 K.

TABLE 1
Form F of Compound (I)
PXRD 2θ values (CuKα)

| | | | | | | |
|---|---|---|---|---|---|---|
| 3.3 ± 0.2° | 6.6 ± 0.2° | 7.7 ± 0.2° | 9.0 ± 0.2° | 11.2 ± 0.2° | 11.9 ± 0.2° | 12.5 ± 0.2° |
| 13.9 ± 0.2° | 15.0 ± 0.2° | 15.4 ± 0.2° | 16.1 ± 0.2° | 16.4 ± 0.2° | 17.6 ± 0.2° | 18.0 ± 0.2° |
| 18.4 ± 0.2° | 18.9 ± 0.2° | 20.1 ± 0.2° | 22.1 ± 0.2° | 22.7 ± 0.2° | 23.2 ± 0.2° | 23.8 ± 0.2° |
| 24.3 ± 0.2° | 25.3 ± 0.2° | 25.5 ± 0.2° | 26.2 ± 0.2° | 26.5 ± 0.2° | 27.2 ± 0.2° | 27.4 ± 0.2° |
| 30.4 ± 0.2° | 32.5 ± 0.2° | — | — | — | — | — |

In some embodiments, crystalline Form F of Compound (I) is characterized by a powder X-ray diffraction (PXRD) pattern comprising two or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In some embodiments, crystalline Form F of Compound (I) is characterized by a powder X-ray diffraction (PXRD) pattern comprising three or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In some embodiments, crystalline Form F of Compound (I) is characterized by a powder X-ray diffraction (PXRD) pattern comprising four or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In some embodiments, crystalline Form F of Compound (I) is characterized by a powder X-ray diffraction (PXRD) pattern comprising five or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In some embodiments, crystalline Form F of Compound (I) is characterized by a powder X-ray diffraction (PXRD) pattern comprising six or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In some embodiments, crystalline Form F of Compound (I) is characterized by a powder X-ray diffraction (PXRD) pattern comprising seven or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

In certain embodiments, crystalline Form F of Compound (I) is characterized by a powder X-ray diffraction (PXRD) pattern comprising 2θ values in degrees (CuKα) at 3.3±0.2 and 7.7±0.2, wherein the PXRD pattern of Form F is measured at room temperature. In further embodiments, the PXRD pattern further comprises one or more 2θ values in degrees selected from: 6.6±0.2, 9.0±0.2, 11.2±0.2, 15.0±0.2, 18.9±0.2, 20.1±0.2, 23.8±0.2, 25.3±0.2, and 27.2±0.2. For example, in some embodiments, crystalline Form F of Compound (I) is characterized by a powder X-ray diffraction (PXRD) pattern comprising 2θ values in degrees (CuKα) at 3.3±0.2, 7.7±0.2, and 9.0±0.2, wherein the PXRD pattern of Form F is measured at room temperature.

In certain embodiments, crystalline Form F of Compound (I) is characterized by a powder X-ray diffraction (PXRD) pattern comprising 2θ values in degrees (CuKα) at 3.3±0.2, 7.7±0.2, and 25.3±0.2, wherein the PXRD pattern of Form F is measured at room temperature.

In further embodiments, crystalline Form F of Compound (I) is characterized by a powder X-ray diffraction (PXRD) pattern comprising 2θ values in degrees (CuKα) at 3.3±0.2, 7.7±0.2, 23.8±0.2, and 25.3±0.2, wherein the PXRD pattern of Form F is measured at room temperature.

In further embodiments, crystalline Form F of Compound (I) is characterized by a powder X-ray diffraction (PXRD) pattern comprising 2θ values in degrees (CuKα) at 3.3±0.2, 7.7±0.2, 9.0±0.2, 23.8±0.2, and 25.3±0.2, wherein the PXRD pattern of Form F is measured at room temperature.

In further embodiments, crystalline Form F of Compound (I) is characterized by a powder X-ray diffraction (PXRD) pattern comprising 2θ values in degrees (CuKα) at 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of Form F is measured at room temperature.

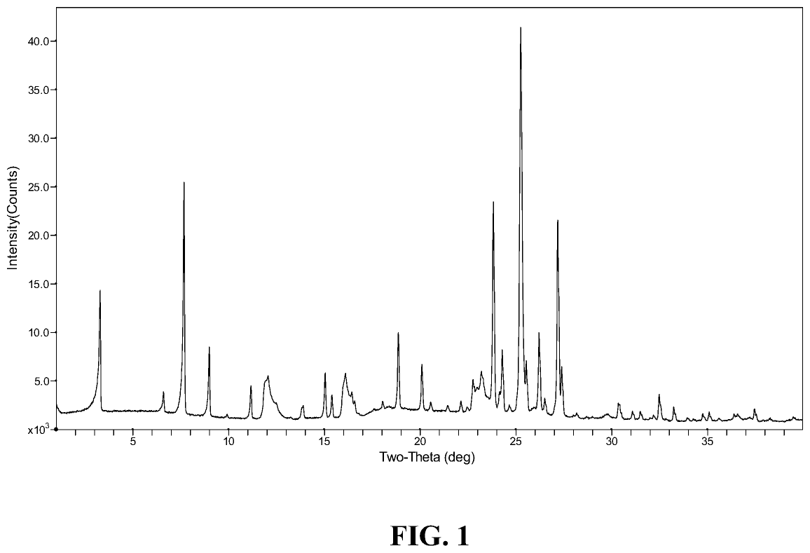

In certain embodiments, crystalline Form F of Compound (I) is characterized by an observed powder X-ray diffraction pattern (CuKα, measured at room temperature) substantially as shown in FIG. 1.

In some embodiments, the Form F of Compound (I) is substantially pure. For example, Form F of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form F. For such embodiments, crystalline Compound (I) may comprise at least about 90 weight %, preferably at least about 95 weight %, and more preferably at least about 99 weight %, of crystalline Form F, based on the weight of Compound (I).

Certain embodiments also provide a composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form F.

In further embodiments, a pharmaceutical composition is provided comprising Form F of Compound (I), and at least one pharmaceutically-acceptable carrier and/or diluent.

In certain embodiments, a pharmaceutical composition comprises substantially pure Form F of Compound (I), and at least one pharmaceutically-acceptable carrier and/or diluent.

In certain embodiments, Form F of Compound (I) is combined with at least one pharmaceutically acceptable carrier and/or diluent to provide at least one pharmaceutical composition.

In some embodiments, a pharmaceutical composition comprises Form F of Compound (I) and other solid forms of Compound (I). Such other solid forms can be, for example, other crystalline forms (e.g., Form A, Form G, Form J, Form K) and/or amorphous Compound (I).

### Form G of Compound (I)

In some embodiments, Compound (I) is provided as a crystalline material comprising Form G. The crystalline Form G of Compound (I) is a free base hydrate crystalline form.

TABLE 2
Form G of Compound (I)
PXRD 2θ values (CuKα)

| | | | | | | |
|---|---|---|---|---|---|---|
| 3.2 ± 0.20 | 5.6 ± 0.2° | 8.6 ± 0.2° | 11.7 ± 0.2° | 14.2 ± 0.2° | 15.0 ± 0.2° | 16.3 ± 0.2° |
| 17.3 ± 0.20 | 18.2 ± 0.2° | 19.9 ± 0.2° | 21.5 ± 0.2° | 23.8 ± 0.2° | 24.3 ± 0.2° | — |

In some embodiments, crystalline Form G of Compound (I) is characterized by a powder X-ray diffraction (PXRD) pattern comprising two or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of Form G is measured at room temperature.

In some embodiments, crystalline Form G of Compound (I) is characterized by a powder X-ray diffraction (PXRD) pattern comprising three or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of Form G is measured at room temperature.

In some embodiments, crystalline Form G of Compound (I) is characterized by a powder X-ray diffraction (PXRD) pattern comprising four or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of Form G is measured at room temperature.

In some embodiments, crystalline Form G of Compound (I) is characterized by a powder X-ray diffraction (PXRD) pattern comprising five or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of Form G is measured at room temperature.

In certain embodiments, crystalline Form G of Compound (I) is characterized by a powder X-ray diffraction (PXRD) pattern comprising 2θ values in degrees (CuKα) at 3.2±0.2 and 5.610.2, wherein the PXRD pattern of Form G is measured at room temperature. In further embodiments, the PXRD pattern further comprises one or more 2θ values in degrees selected from: 8.6±0.2, 11.7±0.2, 14.2±0.2, 15.0±0.2, 17.3=0.2, and 18.2±0.2.

In certain embodiments, crystalline Form G of Compound (I) is characterized by a powder X-ray diffraction (PXRD) pattern comprising 2θ values in degrees (CuKα) at 5.6±0.2 and 8.6±0.2, wherein the PXRD pattern of Form G is measured at room temperature.

In further embodiments, crystalline Form G of Compound (I) is characterized by a powder X-ray diffraction (PXRD) pattern comprising 2θ values in degrees (CuKα) at 3.2±0.2, 5.6±0.2, and 8.6±0.2, wherein the PXRD pattern of Form G is measured at room temperature.

In further embodiments, crystalline Form G of Compound (I) is characterized by a powder X-ray diffraction (PXRD) pattern comprising 2θ values in degrees (CuKα) at 3.2±0.2, 5.6±0.2, 8.6±0.2, and 14.2±0.2, wherein the PXRD pattern of Form G is measured at room temperature.

In further embodiments, crystalline Form G of Compound (I) is characterized by a powder X-ray diffraction (PXRD) pattern comprising 2θ values in degrees (CuKα) at 3.2±0.2, 5.6±0.2, 8.6±0.2, 11.7±0.2, 14.2±0.2, 15.0±0.2, 17.3±0.2, and 18.2±0.2, wherein the PXRD pattern of Form G is measured at room temperature.

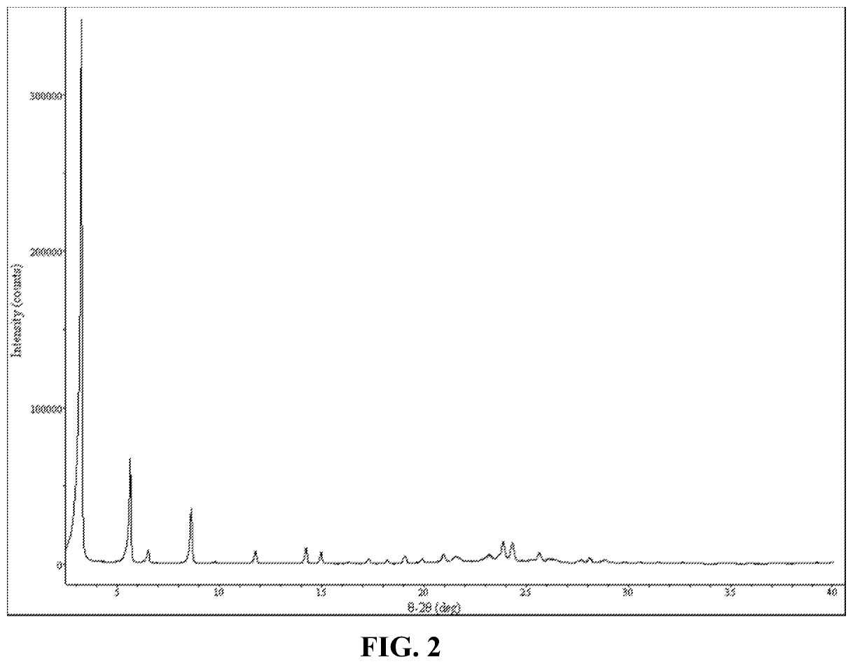

In certain embodiments, crystalline Form G of Compound (I) is characterized by an observed powder X-ray diffraction pattern (CuKα, measured at room temperature) substantially as shown in FIG. 2.

In some embodiments, the Form G of Compound (I) is substantially pure. For example, Form G of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form G. For such embodiments, crystalline Compound (I) may comprise at least about 90 weight %, preferably at least about 95 weight %, and more preferably at least about 99 weight %, of crystalline Form G, based on the weight of Compound (I).

Certain embodiments also provide a composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form G.

In further embodiments, a pharmaceutical composition is provided comprising Form G of Compound (I), and at least one pharmaceutically-acceptable carrier and/or diluent.

In certain embodiments, a pharmaceutical composition comprises substantially pure Form G of Compound (I), and at least one pharmaceutically-acceptable carrier and/or diluent.

In certain embodiments, Form G of Compound (I) is combined with at least one pharmaceutically acceptable carrier and/or diluent to provide at least one pharmaceutical composition.

In some embodiments, a pharmaceutical composition comprises Form G of Compound (I) and other solid forms of Compound (I). Such other solid forms can be, for example, other crystalline forms (e.g., Form F) and/or amorphous Compound (I).

### Form H of Compound (I)

In some embodiments, Compound (I) is provided as a crystalline material comprising Form H. The crystalline Form H of Compound (I) is a free base hydrate crystalline form.

TABLE 3
Form H of Compound (I)
PXRD 2θ values (CuKα)

| | | | | | | |
|---|---|---|---|---|---|---|
| 8.2 ± 0.2° | 9.0 ± 0.2° | 11.4 ± 0.2° | 13.0 ± 0.2° | 13.8 ± 0.2° | 14.7 ± 0.2° | 16.3 ± 0.2° |
| 19.1 ± 0.2° | 19.4 ± 0.2° | 20.0 ± 0.2° | 21.2 ± 0.2° | 21.4 ± 0.2° | 22.1 ± 0.2° | 22.4 ± 0.2° |
| 23.2 ± 0.2° | 24.4 ± 0.2° | 25.0 ± 0.2° | 27.8 ± 0.2° | 28.3 ± 0.2° | 29.1 ± 0.2° | — |

In some embodiments, crystalline Form H of Compound (I) is characterized by a powder X-ray diffraction (PXRD) pattern comprising two or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of Form His measured at room temperature.

In some embodiments, crystalline Form H of Compound (I) is characterized by a powder X-ray diffraction (PXRD) pattern comprising three or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of Form His measured at room temperature.

In some embodiments, crystalline Form H of Compound (I) is characterized by a powder X-ray diffraction (PXRD) pattern comprising four or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of Form His measured at room temperature.

In some embodiments, crystalline Form H of Compound (I) is characterized by a powder X-ray diffraction (PXRD) pattern comprising five or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of Form His measured at room temperature.

In certain embodiments, crystalline Form H of Compound (I) is characterized by a powder X-ray diffraction (PXRD) pattern comprising 2θ values in degrees (CuKα) at 8.2±0.2 and 13.8±0.2, wherein the PXRD pattern of Form H is measured at room temperature. In further embodiments, the PXRD pattern further comprises one or more 2θ values in degrees selected from: 9.0±0.2, 21.2±0.2, 22.4±0.2, 23.2±0.2, 24.4±0.2, 25.0±0.2, and 27.8±0.2.

In certain embodiments, crystalline Form H of Compound (I) is characterized by a powder X-ray diffraction (PXRD) pattern comprising 2θ values in degrees (CuKα) at 8.2±0.2 and 21.2±0.2, wherein the PXRD pattern of Form H is measured at room temperature. In further embodiments, the PXRD pattern further comprises one or more 2θ values in degrees selected from: 9.0±0.2, 13.8±0.2, 22.4±0.2, 23.2±0.2, 24.4±0.2, 25.0±0.2, and 27.8±0.2.

In further embodiments, crystalline Form H of Compound (I) is characterized by a powder X-ray diffraction (PXRD) pattern comprising 2θ values in degrees (CuKα) at 8.2±0.2, 21.2±0.2, and 24.4±0.2, wherein the PXRD pattern of Form His measured at room temperature.

In certain embodiments, crystalline Form H of Compound (I) is characterized by a powder X-ray diffraction (PXRD) pattern comprising 2θ values in degrees (CuKα) at 8.2±0.2, 9.0±0.2, and 13.8±0.2, wherein the PXRD pattern of Form His measured at room temperature.

In further embodiments, crystalline Form H of Compound (I) is characterized by a powder X-ray diffraction (PXRD) pattern comprising 2θ values in degrees (CuKα) at 8.2±0.2, 9.0±0.2, 13.8±0.2, and 21.2±0.2, wherein the PXRD pattern of Form H is measured at room temperature.

In further embodiments, crystalline Form H of Compound (I) is characterized by a powder X-ray diffraction (PXRD) pattern comprising 2θ values in degrees (CuKα) at 8.2±0.2, 9.0±0.2, 13.8±0.2, 21.2±0.2, and 24.4±0.2, wherein the PXRD pattern of Form H is measured at room temperature.

In further embodiments, crystalline Form H of Compound (I) is characterized by a powder X-ray diffraction (PXRD) pattern comprising 2θ values in degrees (CuKα) at 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of Form H is measured at room temperature.

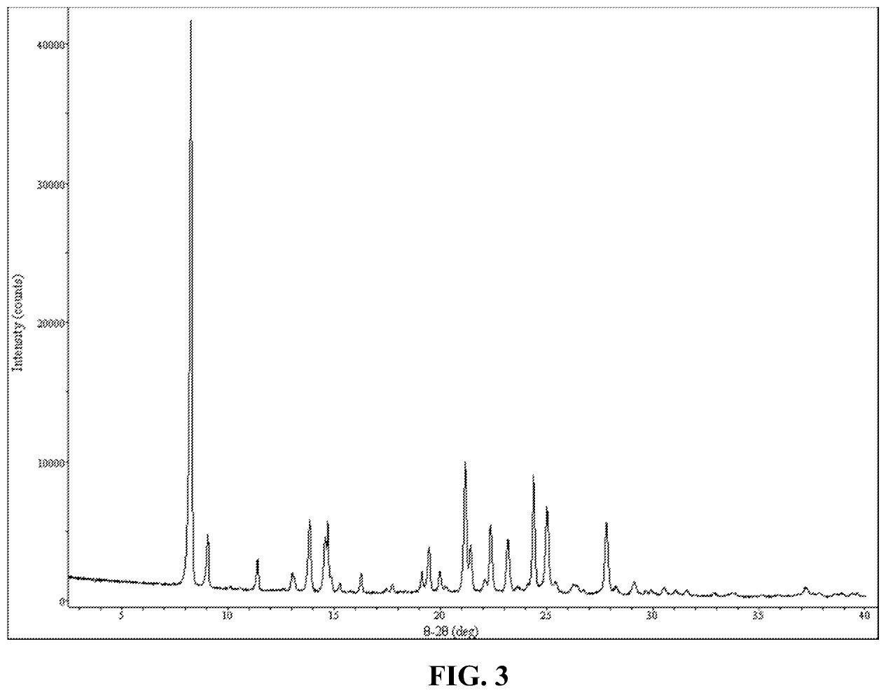

In certain embodiments, crystalline Form H of Compound (I) is characterized by an observed powder X-ray diffraction pattern (CuKα, measured at room temperature) substantially as shown in FIG. 3.

In some embodiments, the Form H of Compound (I) is substantially pure. For example, Form H of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form H. For such embodiments, crystalline Compound (I) may comprise at least about 90 weight %, preferably at least about 95 weight %, and more preferably at least about 99 weight %, of crystalline Form H, based on the weight of Compound (I).

Certain embodiments also provide a composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form H.

In further embodiments, a pharmaceutical composition is provided comprising Form H of Compound (I), and at least one pharmaceutically-acceptable carrier and/or diluent.

In certain embodiments, a pharmaceutical composition comprises substantially pure Form H of Compound (I), and at least one pharmaceutically-acceptable carrier and/or diluent.

In certain embodiments, Form H of Compound (I) is combined with at least one pharmaceutically acceptable carrier and/or diluent to provide at least one pharmaceutical composition.

In some embodiments, a pharmaceutical composition comprises Form H of Compound (I) and other solid forms of Compound (I). Such other solid forms can be, for example, other crystalline forms and/or amorphous Compound (I).

### Form I of Compound (I)

In some embodiments, Compound (I) is provided as a crystalline material comprising Form I. The crystalline Form I of Compound (I) is a free base hydrate crystalline form.

TABLE 4
Form I of Compound (I)
PXRD 2θ values (CuKα)

| | | | | | | |
|---|---|---|---|---|---|---|
| 3.3 ± 0.2° | 5.8 ± 0.2° | 6.7 ± 0.2° | 8.9 ± 0.2° | 12.1 ± 0.2° | 13.4 ± 0.2° | 14.6 ± 0.2° |
| 15.4 ± 0.2° | 16.8 ± 0.2° | 17.8 ± 0.2° | 18.8 ± 0.2° | 20.2 ± 0.2° | 21.4 ± 0.2° | 22.1 ± 0.2° |
| 23.0 ± 0.2° | 23.7 ± 0.2° | — | — | — | — | — |

In some embodiments, crystalline Form I of Compound (I) is characterized by a powder X-ray diffraction (PXRD) pattern comprising two or more 2θ values in degrees (CuKα) selected from: 3.3±0.2, 5.8±0.2, 8.9±0.2, and 14.6±0.2, wherein the PXRD pattern of Form I is measured at room temperature.

In some embodiments, crystalline Form I of Compound (I) is characterized by a powder X-ray diffraction (PXRD) pattern comprising three or more 2θ values in degrees (CuKα) selected from: 3.3±0.2, 5.8±0.2, 8.9±0.2, and 14.6±0.2, wherein the PXRD pattern of Form I is measured at room temperature.

In certain embodiments, crystalline Form I of Compound (I) is characterized by a powder X-ray diffraction (PXRD) pattern comprising 2θ values in degrees (CuKα) at 3.3±0.2 and 5.8±0.2, wherein the PXRD pattern of Form I is measured at room temperature.

In further embodiments, crystalline Form I of Compound (I) is characterized by a powder X-ray diffraction (PXRD) pattern comprising 2θ values in degrees (CuKα) at 3.3±0.2, 5.8±0.2, and 8.9±0.2, wherein the PXRD pattern of Form I is measured at room temperature.

In certain embodiments, crystalline Form I of Compound (I) is characterized by a powder X-ray diffraction (PXRD) pattern comprising 2θ values in degrees (CuKα) at 5.8±0.2 and 8.9±0.2, wherein the PXRD pattern of Form I is measured at room temperature.

In further embodiments, crystalline Form I of Compound (I) is characterized by a powder X-ray diffraction (PXRD) pattern comprising 2θ values in degrees (CuKα) at 5.8±0.2, 8.9±0.2, and 14.6±0.2, wherein the PXRD pattern of Form I is measured at room temperature.

In further embodiments, crystalline Form I of Compound (I) is characterized by a powder X-ray diffraction (PXRD) pattern comprising 2θ values in degrees (CuKα) at 3.3±0.2, 5.8±0.2, 8.9±0.2, and 14.6±0.2, wherein the PXRD pattern of Form I is measured at room temperature.

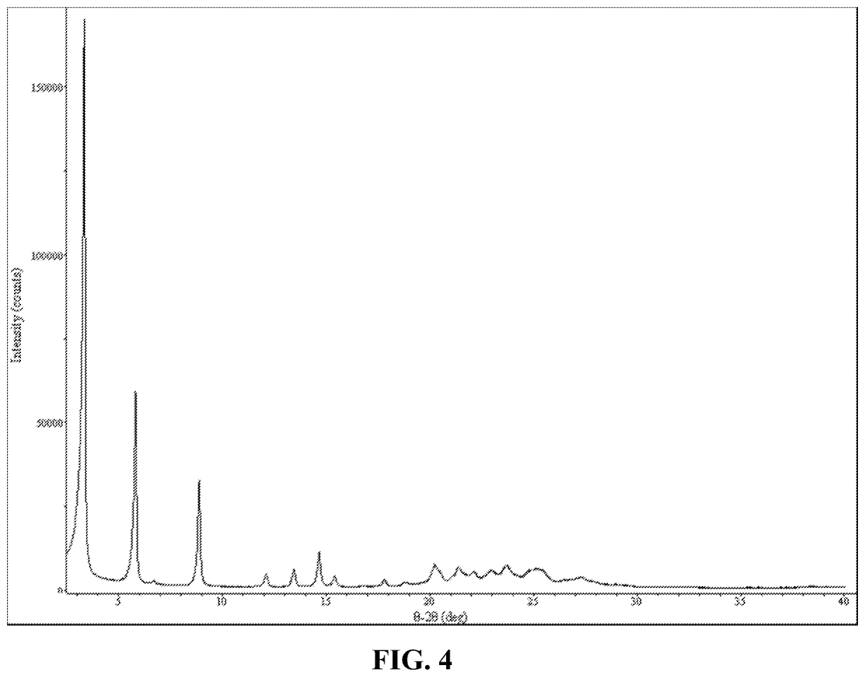

In certain embodiments, crystalline Form I of Compound (I) is characterized by an observed powder X-ray diffraction pattern (CuKα, measured at room temperature) substantially as shown in FIG. 4.

In some embodiments, the Form I of Compound (I) is substantially pure. For example, Form I of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form I. For such embodiments, crystalline Compound (I) may comprise at least about 90 weight %, preferably at least about 95 weight %, and more preferably at least about 99 weight %, of crystalline Form I, based on the weight of Compound (I).

Certain embodiments also provide a composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form I.

In further embodiments, a pharmaceutical composition is provided comprising Form I of Compound (I), and at least one pharmaceutically-acceptable carrier and/or diluent.

In certain embodiments, a pharmaceutical composition comprises substantially pure Form I of Compound (I), and at least one pharmaceutically-acceptable carrier and/or diluent.

In certain embodiments, Form I of Compound (I) is combined with at least one pharmaceutically acceptable carrier and/or diluent to provide at least one pharmaceutical composition.

In some embodiments, a pharmaceutical composition comprises Form I of Compound (I) and other solid forms of Compound (I). Such other solid forms can be, for example, other crystalline forms (e.g., Form G) and/or amorphous Compound (I).

### Form J of Compound (I)

In some embodiments, Compound (I) is provided as a crystalline material comprising Form J. The crystalline Form J of Compound (I) is a free base anhydrous crystalline form.

TABLE 5
Form J of Compound (I)
PXRD 2θ values (CuKα)

| | | | | | | |
|---|---|---|---|---|---|---|
| 4.0 ± 0.2° | 7.4 ± 0.2° | 8.1 ± 0.2° | 8.7 ± 0.2° | 11.4 ± 0.2° | 12.0 ± 0.2° | 12.1 ± 0.2° |
| 12.6 ± 0.2° | 12.8 ± 0.2° | 13.5 ± 0.2° | 14.8 ± 0.2° | 16.0 ± 0.2° | 16.2 ± 0.2° | 16.8 ± 0.2° |
| 17.5 ± 0.2° | 18.4 ± 0.2° | 19.4 ± 0.2° | 19.9 ± 0.2° | 20.8 ± 0.2° | 22.0 ± 0.2° | 22.4 ± 0.2° |
| 23.2 ± 0.2° | 23.5 ± 0.2° | 24.4 ± 0.2° | 25.4 ± 0.2° | 25.8 ± 0.2° | 26.7 ± 0.2° | 27.3 ± 0.2° |
| 27.6 ± 0.2° | 28.4 ± 0.2° | 28.8 ± 0.2° | 29.1 ± 0.2° | — | — | — |

In some embodiments, crystalline Form J of Compound (I) is characterized by a powder X-ray diffraction (PXRD) pattern comprising two or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

In some embodiments, crystalline Form J of Compound (I) is characterized by a powder X-ray diffraction (PXRD) pattern comprising three or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

In some embodiments, crystalline Form J of Compound (I) is characterized by a powder X-ray diffraction (PXRD) pattern comprising four or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

In some embodiments, crystalline Form J of Compound (I) is characterized by a powder X-ray diffraction (PXRD) pattern comprising five or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

In some embodiments, crystalline Form J of Compound (I) is characterized by a powder X-ray diffraction (PXRD) pattern comprising six or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

In some embodiments, crystalline Form J of Compound (I) is characterized by a powder X-ray diffraction (PXRD) pattern comprising seven or more 2θ values in degrees (CuKα) selected from: 4.0±0.2, 7.4±0.2, 8.1±0.2, 8.7±0.2, 11.4±0.2, 14.8=0.2, 16.0±0.2, 16.2±0.2, 23.2±0.2, 23.5±0.2, 24.4±0.2, and 25.8±0.2, wherein the PXRD pattern of Form J is measured at room temperature.

In certain embodiments, crystalline Form J of Compound (I) is characterized by a powder X-ray diffraction (PXRD) pattern comprising 2θ values in degrees (CuKα) at 7.4±0.2 and 8.1±0.2, wherein the PXRD pattern of Form J is measured at room temperature. In further embodiments, the PXRD pattern further comprises one or more 2θ values in degrees selected from: 4.0±0.2, 8.7±0.2, 11.4±0.2, 14.8±0.2, 16.0±0.2, 16.2±0.2, 23.2±0.2, 23.5±0.2, 24.4±0.2, and 25.8±0.2.

In certain embodiments, crystalline Form J of Compound (I) is characterized by a powder X-ray diffraction (PXRD) pattern comprising 2θ values in degrees (CuKα) at 7.4±0.2, 8.1±0.2, and 8.7±0.2, wherein the PXRD pattern of Form J is measured at room temperature.

In certain embodiments, crystalline Form J of Compound (I) is characterized by a powder X-ray diffraction (PXRD) pattern comprising 2θ values in degrees (CuKα) at 7.4±0.2, 8.1±0.2, and 16.0±0.2, wherein the PXRD pattern of Form J is measured at room temperature.

In further embodiments, crystalline Form J of Compound (I) is characterized by a powder X-ray diffraction (PXRD) pattern comprising 2θ values in degrees (CuKα) at 7.4±0.2, 8.1±0.2, 16.0±0.2, and 23.5±0.2, wherein the PXRD pattern of Form J is measured at room temperature.

In further embodiments, crystalline Form J of Compound (I) is characterized by a powder X-ray diffraction (PXRD) pattern comprising 2θ values in degrees (CuKα) at 7.4±0.2, 8.1±0.2, 16.0±0.2, 23.5±0.2, and 24.4±0.2, wherein the PXRD pattern of Form J is measured at room temperature.

In further embodiments, crystalline Form J of Compound (I) is characterized by a powder X-ray diffraction (PXRD) pattern comprising 2θ values in degrees (CuKα) at 7.4±0.2, 8.1±0.2, 8.7±0.2, 16.0±0.2, 23.5±0.2, and 24.4±0.2, wherein the PXRD pattern of Form J is measured at room temperature.

In further embodiments, crystalline Form J of Compound (I) is characterized by a powder X-ray diffraction (PXRD) pattern comprising 2θ values in degrees (CuKα) at 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature.

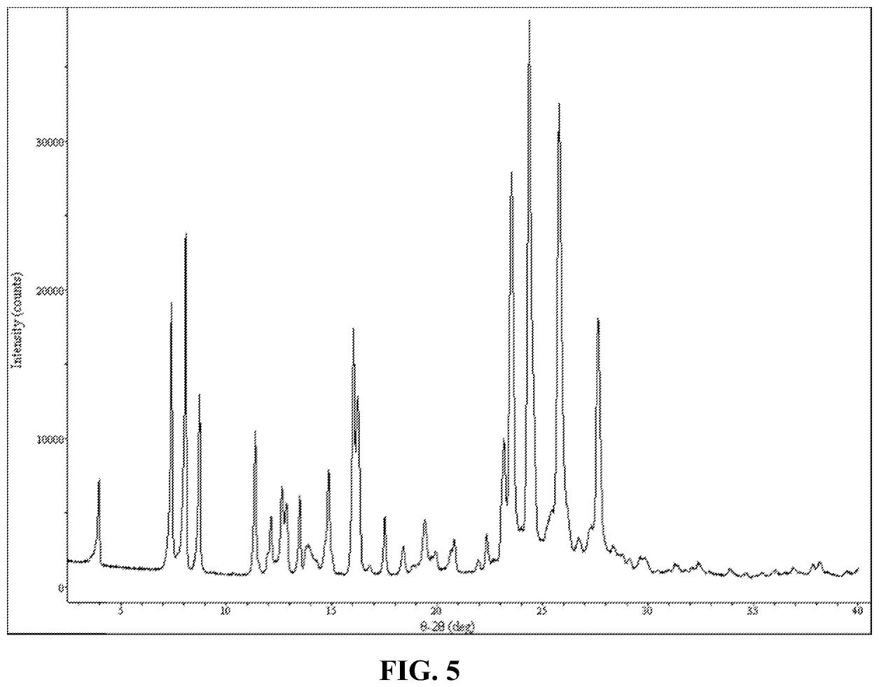

In certain embodiments, crystalline Form J of Compound (I) is characterized by an observed powder X-ray diffraction pattern (CuKα, measured at room temperature) substantially as shown in FIG. 5.

In some embodiments, crystalline Form J of Compound (I) is characterized by an endotherm with a peak maximum in the approximate range of from 261° C. to 265° C. In further embodiments, the endotherm maximum is at about 263° C. For example, in some embodiments, crystalline Form J of Compound (I) is characterized by a differential scanning calorimetry (DSC) thermogram comprising an endotherm with a maximum in the range of from about 261° C. to about 265° C.; in certain embodiments, the differential scanning calorimetry (DSC) thermogram comprises an endotherm with a maximum at about 263° C. It should be understood that, in some cases, the endothermic event may not be detected.

In certain embodiments, crystalline Form J of Compound (I) is characterized by (i) a powder X-ray diffraction (PXRD) pattern comprising 2θ values in degrees (CuKα) at 7.4±0.2 and 8.1±0.2, wherein the PXRD pattern of Form J is measured at room temperature; and (ii) an endotherm with a peak maximum in the approximate range of from 261° C. to 265° C. In further embodiments, the endotherm peak maximum is at about 263° C.

In certain embodiments, crystalline Form J of Compound (I) is characterized by (i) a powder X-ray diffraction (PXRD) pattern comprising 2θ values in degrees (CuKα) at 7.4±0.2, 8.1±0.2, 8.7±0.2, and 16.0±0.2, wherein the PXRD pattern of Form J is measured at room temperature; and (ii) an endotherm with peak max in the approximate range of from 261° C. to 265° C. In further embodiments, the endotherm peak maximum is at about 263° C.

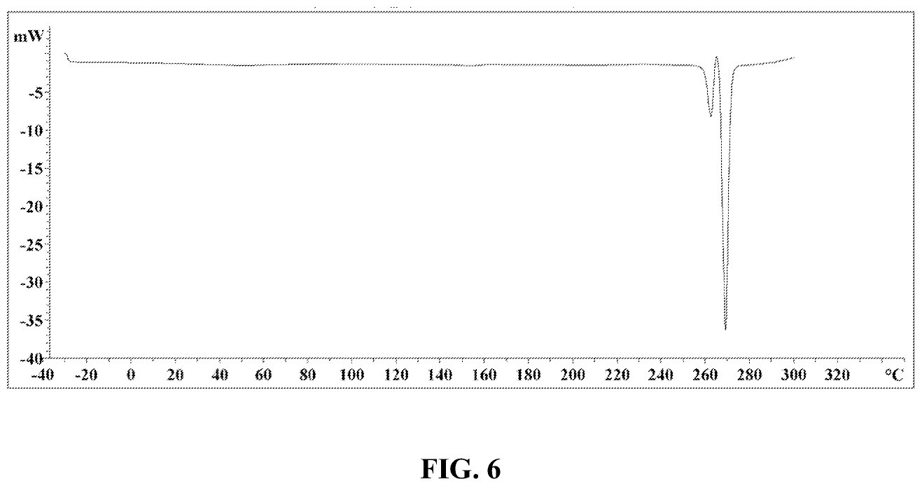

In some embodiments, crystalline Form J of Compound (I) is characterized by a differential scanning calorimetry (DSC) thermogram substantially in accordance with the thermogram shown in FIG. 6.

In certain embodiments, crystalline Form J of Compound (I) is characterized by (i) a powder X-ray diffraction (PXRD) pattern comprising two or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of Form J is measured at room temperature; and (ii) a differential scanning calorimetry (DSC) thermogram substantially in accordance with the thermogram shown in FIG. 6.

In some embodiments, crystalline Form J of Compound (I) is characterized by a thermogravimetric analysis (TGA) thermogram having weight loss of less than 0.1% upon being heated (e.g., from room temperature) to a temperature of about 150° C. In certain embodiments, crystalline Form J of Compound (I) is characterized by a thermogravimetric analysis (TGA) thermogram having weight loss of less than 0.1% upon being heated to a temperature of about 175° C. In further embodiments, crystalline Form J of Compound (I) is characterized by a thermogravimetric analysis (TGA) thermogram showing less than about 0.5% weight loss, and in certain embodiments less than about 0.1% weight loss, at about 200° C.

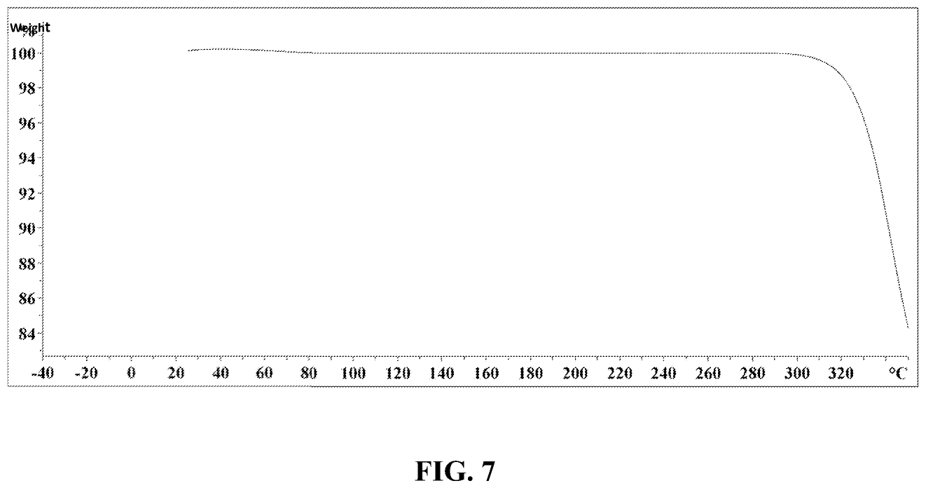

In certain embodiments, crystalline Form J of Compound (I) exhibits a thermogravimetric analysis (TGA) thermogram substantially as shown in FIG. 7.

In some embodiments, the Form J of Compound (I) is substantially pure. For example, Form J of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form J. For such embodiments, crystalline Compound (I) may comprise at least about 90 weight %, preferably at least about 95 weight %, and more preferably at least about 99 weight %, of crystalline Form J, based on the weight of Compound (I).

Certain embodiments also provide a composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl)amino)-N-(methyl-d₃)pyridazine-3-carboxamide is in crystalline Form J.

In further embodiments, a pharmaceutical composition is provided comprising Form J of Compound (I), and at least one pharmaceutically-acceptable carrier and/or diluent.

In certain embodiments, a pharmaceutical composition comprises substantially pure Form J of Compound (I), and at least one pharmaceutically-acceptable carrier and/or diluent.

In certain embodiments, Form J of Compound (I) is combined with at least one pharmaceutically acceptable carrier and/or diluent to provide at least one pharmaceutical composition.

In some embodiments, a pharmaceutical composition comprises Form J of Compound (I) and other solid forms of Compound (I). Such other solid forms can be, for example, other crystalline forms (e.g., Form F) and/or amorphous Compound (I).

### Form K of Compound (I)

In some embodiments, Compound (I) is provided as a crystalline material comprising Form K. The crystalline Form K of Compound (I) is a free base monohydrate crystalline form.

TABLE 6
Form K of Compound (I)
PXRD 2θ values (CuKα)

| | | | | | | |
|---|---|---|---|---|---|---|
| 3.8 ± 0.2° | 7.7 ± 0.2° | 9.4 ± 0.2° | 11.4 ± 0.2° | 12.1 ± 0.2° | 12.4 ± 0.2° | 12.9 ± 0.2° |
| 13.8 ± 0.2° | 15.0 ± 0.2° | 15.2 ± 0.2° | 15.5 ± 0.2° | 16.1 ± 0.2° | 16.3 ± 0.2° | 16.7 ± 0.2° |
| 17.4 ± 0.2° | 18.2 ± 0.2° | 18.9 ± 0.2° | 19.2 ± 0.2° | 20.3 ± 0.2° | 21.4 ± 0.2° | 22.3 ± 0.2° |
| 22.6 ± 0.2° | 22.9 ± 0.2° | 23.3 ± 0.2° | 23.6 ± 0.2° | 23.8 ± 0.2° | 24.3 ± 0.2° | 25.7 ± 0.2° |
| 26.5 ± 0.2° | 27.7 ± 0.2° | 28.5 ± 0.2° | — | — | — | — |

In some embodiments, crystalline Form K of Compound (I) is characterized by a powder X-ray diffraction (PXRD) pattern comprising two or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In some embodiments, crystalline Form K of Compound (I) is characterized by a powder X-ray diffraction (PXRD) pattern comprising three or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In some embodiments, crystalline Form K of Compound (I) is characterized by a powder X-ray diffraction (PXRD) pattern comprising four or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In some embodiments, crystalline Form K of Compound (I) is characterized by a powder X-ray diffraction (PXRD) pattern comprising five or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In some embodiments, crystalline Form K of Compound (I) is characterized by a powder X-ray diffraction (PXRD) pattern comprising six or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In some embodiments, crystalline Form K of Compound (I) is characterized by a powder X-ray diffraction (PXRD) pattern comprising seven or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature.

In certain embodiments, crystalline Form K of Compound (I) is characterized by a powder X-ray diffraction (PXRD) pattern comprising 2θ values in degrees (CuKα) at 7.7±0.2 and 12.1±0.2, wherein the PXRD pattern of Form K is measured at room temperature. In further embodiments, the PXRD pattern further comprises one or more 2θ values in degrees selected from: 3.8±0.2, 9.4±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2.

In certain embodiments, crystalline Form K of Compound (I) is characterized by a powder X-ray diffraction (PXRD) pattern comprising 2θ values in degrees (CuKα) at 7.7±0.2, 12.1±0.2, and 16.3±0.2, wherein the PXRD pattern of Form K is measured at room temperature.

In certain embodiments, crystalline Form K of Compound (I) is characterized by a powder X-ray diffraction (PXRD) pattern comprising 2θ values in degrees (CuKα) at 3.8±0.2, 7.7±0.2, and 12.1±0.2, wherein the PXRD pattern of Form K is measured at room temperature.

In certain embodiments, crystalline Form K of Compound (I) is characterized by a powder X-ray diffraction (PXRD) pattern comprising 2θ values in degrees (CuKα) at 7.7±0.2, 12.1±0.2, 16.3±0.2, and 24.3±0.2, wherein the PXRD pattern of Form K is measured at room temperature.

In further embodiments, crystalline Form K of Compound (I) is characterized by a powder X-ray diffraction (PXRD) pattern comprising 2θ values in degrees (CuKα) at 7.7±0.2, 12.1±0.2, 16.3±0.2, 18.9±0.2, and 24.3±0.2, wherein the PXRD pattern of Form K is measured at room temperature.

In further embodiments, crystalline Form K of Compound (I) is characterized by a powder X-ray diffraction (PXRD) pattern comprising 2θ values in degrees (CuKα) at 3.8±0.2, 7.7±0.2, 9.4±0.2, 12.1±0.2, 12.4±0.2, 15.5±0.2, 16.1±0.2, 16.3=0.2, 18.9±0.2, 23.6±0.2, 23.8±0.2, 24.3±0.2, 25.7±0.2, and 27.7±0.2, wherein the PXRD pattern of Form K is measured at room temperature.

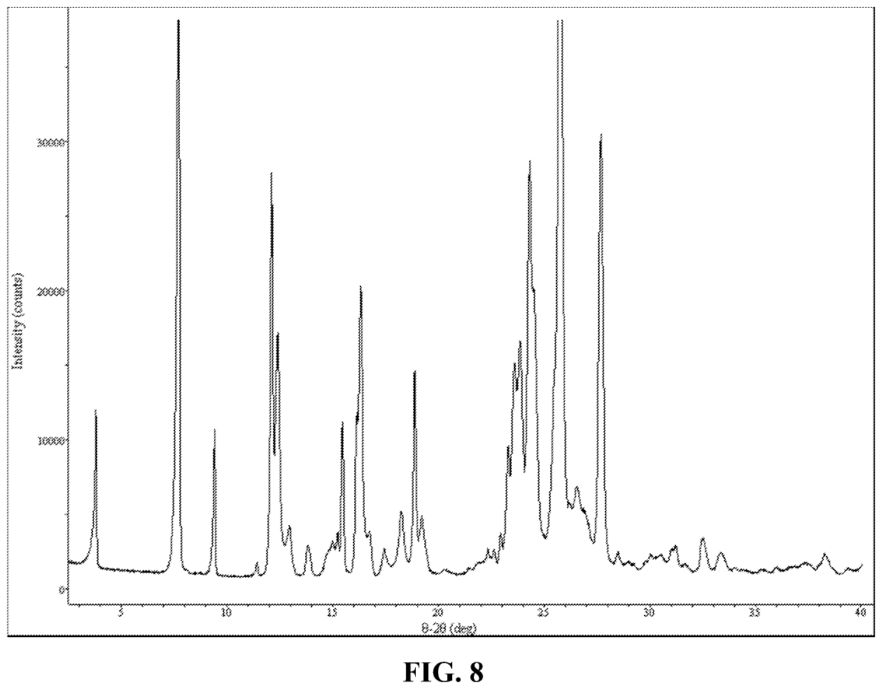

In certain embodiments, crystalline Form K of Compound (I) is characterized by an observed powder X-ray diffraction pattern (CuKα, measured at room temperature) substantially as shown in FIG. 8.

In some embodiments, crystalline Form K of Compound (I) is characterized by an endotherm having a peak maximum below about 130° C. In certain embodiments, the endothermic maximum is from room temperature to about 125° C. For example, in some embodiments, crystalline Form K of Compound (I) is characterized by a differential scanning calorimetry (DSC) thermogram comprising an endotherm with a maximum in the range of from room temperature to about 125° C. It should be understood that, in some cases, the endothermic event may not be detected.

In certain embodiments, crystalline Form K of Compound (I) is characterized by (i) a powder X-ray diffraction (PXRD) pattern comprising 2θ values in degrees (CuKα) at 7.7±0.2 and 12.1±0.2, wherein the PXRD pattern of Form K is measured at room temperature; and (ii) a differential scanning calorimetry (DSC) thermogram comprising an endotherm with a peak from room temperature to about 125° C.

In certain embodiments, crystalline Form K of Compound (I) is characterized by (i) a powder X-ray diffraction (PXRD) pattern comprising 2θ values in degrees (CuKα) at 3.8±0.2, 7.7±0.2, 12.1±0.2, and 16.3±0.2, wherein the PXRD pattern of Form K is measured at room temperature; and (ii) a differential scanning calorimetry (DSC) thermogram comprising an endotherm with a peak from room temperature to about 125° C.

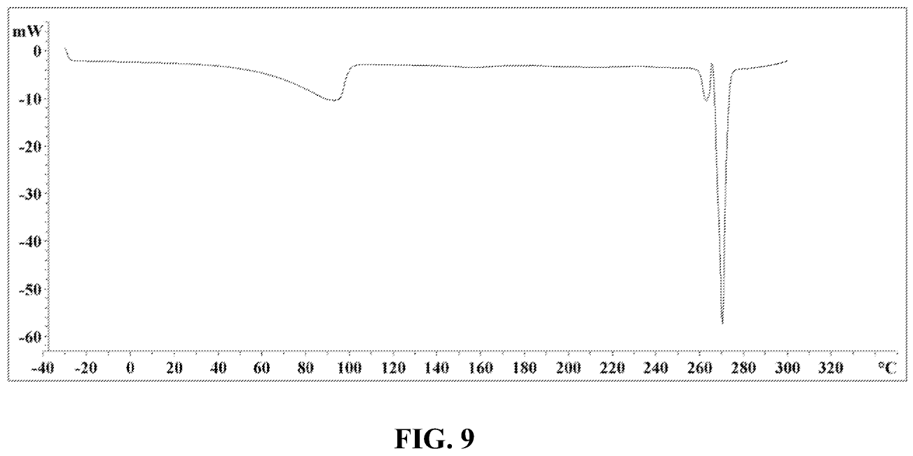

In some embodiments, crystalline Form K of Compound (I) is characterized by a differential scanning calorimetry (DSC) thermogram substantially in accordance with the thermogram shown in FIG. 9.

In certain embodiments, crystalline Form K of Compound (I) is characterized by (i) a powder X-ray diffraction (PXRD) pattern comprising two or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of Form K is measured at room temperature; and (ii) a differential scanning calorimetry (DSC) thermogram substantially in accordance with the thermogram shown in FIG. 9.

In some embodiments, crystalline Form K of Compound (I) is characterized by a thermogravimetric analysis (TGA) thermogram having weight loss of about 4% upon being heated (e.g., from room temperature) to a temperature of about 125° C.

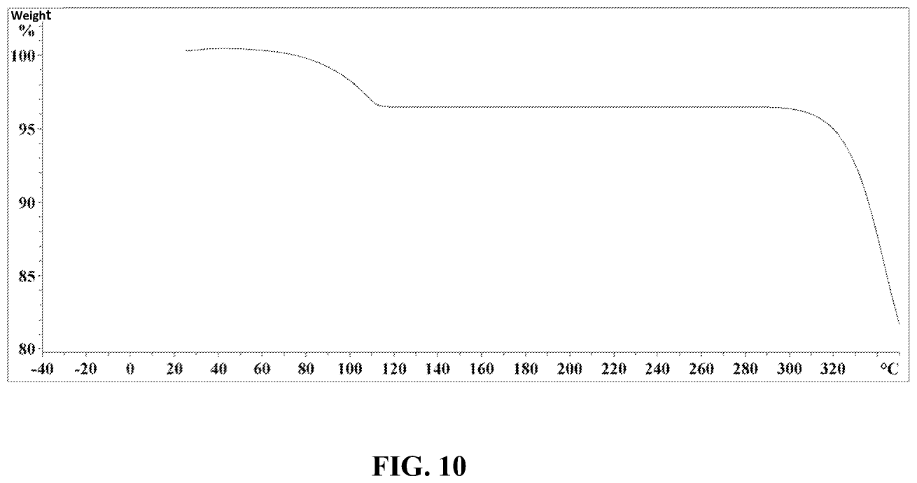

In certain embodiments, crystalline Form K of Compound (I) exhibits a thermogravimetric analysis (TGA) thermogram substantially as shown in FIG. 10.

In some embodiments, the Form K of Compound (I) is substantially pure. For example, Form K of Compound (I) may be present in a sample at a purity of greater than 90 weight %, greater than 95 weight %, or greater than 99 weight %, while the remaining material comprises other form(s) of the compound and/or reaction impurities and/or processing impurities.

In certain embodiments, the crystalline form of Compound (I) consists essentially of Form K. For such embodiments, crystalline Compound (I) may comprise at least about 90 weight %, preferably at least about 95 weight %, and more preferably at least about 99 weight %, of crystalline Form K, based on the weight of Compound (I).

Certain embodiments also provide a composition comprising 6-(cyclopropanecarboxamido)-4-((2-methoxy-3-(1-methyl-1H-1,2,4-triazol-3-yl)phenyl)amino)-N-(methyl-d₃)pyridazine-3-carboxamide, wherein at least 95 weight %, preferably at least 97 weight %, and more preferably at least 99 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form K.

In further embodiments, a pharmaceutical composition is provided comprising Form K of Compound (I), and at least one pharmaceutically-acceptable carrier and/or diluent.

In certain embodiments, a pharmaceutical composition comprises substantially pure Form K of Compound (I), and at least one pharmaceutically-acceptable carrier and/or diluent.

In certain embodiments, Form K of Compound (I) is combined with at least one pharmaceutically acceptable carrier and/or diluent to provide at least one pharmaceutical composition.

In some embodiments, a pharmaceutical composition comprises Form K of Compound (I) and other solid forms of Compound (I). Such other solid forms can be, for example, other crystalline forms (e.g., Form F) and/or amorphous Compound (I).

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Indiana (1999).

For crystallization techniques that employ solvent, the choice of solvent or solvents typically depends on one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971,26, 369-377. In general, seeds of small size are needed to effectively control the growth of crystals in the batch. Seeds of small size may be generated by sieving, milling, or micronizing of large crystals, or by micro-crystallization of solutions. Care should be taken that milling or micronizing of crystals does not result in any change in crystallinity from the desired crystal form (i.e., a change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by one or more suitable spectroscopic or analytical techniques, such as solid state nuclear magnetic resonance, differential scanning calorimetry, X-ray powder diffraction, or the like, to assess formation of the preferred crystalline form of the product. The resulting crystalline form is typically produced in an amount of greater than about 70 weight % isolated yield, preferably greater than 90 weight % isolated yield, based on the weight of the compound originally employed in the crystallization procedure. The product may be co-milled or passed through a mesh screen to delump the product, if necessary.

Crystalline forms may be prepared directly from the reaction medium of the final process for preparing Compound (I). Such preparation may be achieved, for example, by employing in the final process step a solvent or a mixture of solvents from which Compound (I) may be crystallized. Alternatively, crystalline forms may be obtained by distillation or solvent addition techniques. Suitable solvents for this purpose include, for example, the aforementioned nonpolar solvents and polar solvents, including protic polar solvents such as alcohols, and aprotic polar solvents such as ketones.

The presence of more than one polymorph in a sample may be determined by techniques such as powder X-ray diffraction (PXRD) or solid state nuclear magnetic resonance spectroscopy (ssNMR). For example, the presence of extra peaks in the comparison of an experimentally measured PXRD pattern with a simulated PXRD pattern may indicate the presence of more than one polymorph in the sample. The simulated PXRD may be calculated from single crystal X-ray data. See Smith, D. K., “A FORTRAN Program for Calculating X-Ray Powder Diffraction Patterns,” Lawrence Radiation Laboratory, Livermore, California, UCRL-7196 (April 1963).

Form E of Compound (I) may be characterized using various techniques, the operation of which are well known to those of ordinary skill in the art. Form E may be characterized and distinguished using single crystal X-ray diffraction, which is based on unit cell measurements of a single crystal at a fixed analytical temperature. A detailed description of unit cells is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice may be characterized according to the observed fractional atomic coordinates. Another means of characterizing the crystalline structure is by powder X-ray diffraction analysis in which the diffraction profile is compared to a simulated profile representing pure powder material, both run at the same analytical temperature, and measurements for the subject form are characterized as a series of 2θ values (e.g., two, three, four or more).

Other methods of characterizing the form may be used. Such methods include solid state nuclear magnetic resonance (ssNMR), differential scanning calorimetry (DSC), thermography, and gross examination of the crystalline or amorphous morphology. Two or more of these parameters may also be used in combination to characterize the subject form.

### UTILITY

The crystalline forms of Compound (I) described herein can be used to isolate Compound (I) from other components at the completion of the synthesis process; and/or to purify Compound (I) by one or a series of crystallization steps. The isolation and the purification steps can be combined or practiced as separate process steps. Each of the crystalline forms described herein can also be used to make other solid forms of Compound (I), including, for example, Form A (which is described in WO 2018/183656), one or more of the other forms described herein, and/or amorphous Compound (I). For example, in certain embodiments, a method of preparing amorphous Compound (I) comprises preparing crystalline Form F, wherein crystalline Form F is characterized as described herein. In further embodiments, amorphous Compound (I) is then used to make a dosage form (e.g., a dosage form comprising a solid dispersion of amorphous Compound (I)) for clinical use.

Each of the crystalline forms of Compound (I) described herein may be used alone or in combination with other forms of Compound (I) (including the other crystalline forms described herein), and/or formulated with one or more excipients or other active pharmaceutical ingredients to make pharmaceutical compositions.

### EXAMPLES

The invention will now be further described by the following working example(s), which are preferred embodiments of the invention. All temperatures are in degrees Celsius (° C.) unless otherwise indicated. These examples are illustrative rather than limiting and it is to be understood that there may be other embodiments that fall within the spirit and scope of the invention.

For ease of reference, the following abbreviations may be used herein.

### ABBREVIATIONS

| | |
|---|---|
| ACN or MeCN | acetonitrile |
| AP | area percent |
| DBU | 1,8-diazabicyclo[5.4.0]undec-7-ene |
| DIPEA | N,N-diisopropylethylamine (Hunig's base) |
| EDC HCl | 1-(dimethylaminopropyl)-3-ethylcarbodiimide |
| | hydrochloride |
| equiv. | molar equivalents |
| Et | ethyl |
| h | hour(s) |
| HOBt | 1-hydroxy benzotriazole |
| IPA | isopropyl alcohol |
| min | minute(s) |
| Me | methyl |
| MTBE | methyl tert-butyl ether |
| NMP | n-methylpyrrolidone |
| Pd/C | palladium on carbon |
| RH | relative humidity |
| THF | tetrahydrofuran |
| TsCl | p-toluenesulfonyl chloride |

### Example 1: Preparation of Crystalline Form F of Compound (I)

Approximately 80 mg of Compound (I) was dissolved in 2.5 g of isopropanol: water 50:50 (w/w) at 75° C. The solution was hot filtered. Filtered solution was fast cooled to 23° C. in 10 minutes and further fast cooled to 5° C. in 13 minutes, generating white precipitate. The solids were then isolated by vacuum filtration and air-dried. The solids comprised Form F.

In another example, approximately 100 mg of Compound (I) was dissolved in approximately 2 g of an emulsion of 1-butanol: water 80:20 (wt/wt) at 75° C. The solution was hot filtered. Filtered solution was fast cooled to ambient temperature in less than 30 minutes, generating white suspension. The solids were then isolated by vacuum filtration and air-dried. Solids comprised Form F.

In another example, approximately 104 mg of Compound (I) was dissolved in approximately 2 g of an emulsion of 1-butanol: water 80:20 (wt/wt) at 75° C. The solution was hot filtered. The filtered solution was slowly cooled to ambient temperature over 5 hours and allowed to age without agitation overnight. The resulting solids were then isolated by vacuum filtration and air-dried. The solids comprised Form F.

In another preparation, approximately 4 mg of {[(6E)-6-(cyclopropanecarbonylimino)-4-{[2-methoxy-3-(1-methyl-1H-1,2,4-triazol-3-yl)phenyl]amino}-3-[(2H3)methylcarbamoyl]-1,6-dihydropyridazin-1-yl]methoxy }phosphonic acid and 2 molar equivalent of KOH were dissolved in 0.6 mL of NMP:water 50:50 (v/v) by heating the solution up to 50° C. in a 1 dram vial. The vial was partially capped to allow for slow solvent evaporation at room temperature. Crystals were obtained after one week and comprised Form F.

### Example 2: Preparation of Crystalline Form G of Compound (I)

Approximately 2.0 g of Compound (I) were dissolved in 36.0 g of an emulsion of 1-butanol:water 80:20 (wt/wt) at 75° C. The solution was hot filtered in a new pre-warmed container and the filtered solution was slowly cooled to ambient temperature over 5 hours and allowed to age without agitation overnight.

The solids were then isolated by vacuum filtration, washed 2× with 10 mL of acetone:water 70:30 (v/v), air-dried for ˜10 minutes, and equilibrated at 84% RH for 18 days. A sample of the resulting material (4.7 g) was suspended in 20 mL of acetone:water 70:30 (v/v) and subjected to a temperature cycling experiment: 5 cycles at 5-45-(-5) ° C., which produced a thick immobile suspension. 10 mL of acetone:water 70:30 (v/v) was added and the mobile suspension was subjected to a temperature cycling experiment: 2 cycles at 5-45-5° C.; 3 cycles at 5-35-5° C.; and one cycle at 10-35-10° C. The resulting solids were isolated by vacuum filtration and washed 1× with 5 mL of acetone:water 70:30 (v/v), producing 2.6 g of solids. A portion of the latter solids (418.4 mg) was exposed to 84% RH for 24 hours, yielding 324.7 mg of solids. Those solids comprised Form G.

Form G of Compound (I) was also obtained by evaporation of a filtrate in acetone: 1-butanol:water 19:56:25% (w/w/w) at ambient conditions.

Form G of Compound (I) was also prepared by exposure of amorphous material to 75% RH at ambient temperature for 17 up to 46 days.

In another example, 360 mg of Compound (I) was dissolved in 24 mL of tetrahydrofuran:water 95:5 (v/v) at ambient temperature. 71 μL of 37% hydrochloric acid was added. Solids were isolated by quick evaporation, and 90 mg of the isolated solids were suspended in 1 mL of ethanol:water 1:2 (v/v) at 60° C. and agitated overnight. The solids comprised Form G.

In another preparation, 360 mg of Compound (I) was dissolved in 24 mL of tetrahydrofuran:water 95:5 (v/v) at ambient temperature. 55 μL of methanesulfonic acid was added. Solids were isolated by quick evaporation, and 90 mg of the isolated solids were suspended in 1 mL of ethanol:water 1:2 (v/v) at 60° C. and agitated overnight. The suspension was then filtered, and the filtered solids were dried at 50° C. in a vacuum overnight. The dried solids comprised Form G.

### Example 3: Preparation of Crystalline Form H of Compound (I)

Form H of Compound (I) was produced by rotary evaporation in chloroform:methanol 50:50 (v/v), followed by vacuum drying at elevated temperature (in this example, 45° C.) overnight.

Specifically, approximately 100 mg of Compound (I) were dissolved in 2.3 mL of chloroform:methanol 50:50 (v/v). The solution was filtered into a new container and the filtered solution was rotary evaporated to dryness at 40° C. The resulting solids were vacuum dried at 45° C. for 17 hours and stored over a strong desiccant (in this example, P₂O₅), yielding 95.5 mg of dried solids. The solids comprised Form H.

In another preparation, 700 mg of Compound (I) was added to 174.3 grams of 95wt %/5wt % THF/water under magnetic stirring in a flask. After at least 30 minutes of stirring, solids were not fully dissolved, and an additional 40.10 grams of 95wt %/5wt % THF/water was added. After at least another 30 minutes of stirring, insoluble solids were still observed. The solution was then spray dried. The solids comprised a mixture of Form H and Form A (see WO 2018/183656 for a description of Form A).

### Example 4: Preparation of Crystalline Form I of Compound (I)

Form I of Compound (I) was prepared by vacuum drying Form G at elevated temperature (in this example, 45° C. for 24 hours.

Specifically, approximately 108 mg of Form G were vacuum dried at 45° C. for 24 hours and stored over a strong desiccant (in this example, P₂O₅), yielding 64.9 mg of dried solids. The solids comprised Form I.

In another preparation, 100 mg of Compound (I) was dissolved in a mixture of 4.75 mL tetrahydrofuran and 0.25 mL water. 50 μL of this solution was dispensed into a well of a 96-well plate. Solvent was evaporated. Then IPA:water 1:2 (v/v) was added into the well. The plate was sealed and exposed to the following temperature cycling procedure, which was repeated nine times: isothermal at 50° C. for 2 hours; cool to 20° C. over 8 hours in a linear fashion; heat to 50° C. The plate was then unsealed and solvent was evaporated. The solids comprised Form I.

### Example 5: Preparation of Crystalline Form J of Compound (I)

Form J of Compound (I) was prepared as follows: approximately 512.0 mg of wet Form F of Compound (I) were vacuum dried at 40-48° C. for 3 hours and stored over a strong desiccant (in this example, P₂O₅), yielding 161.5 mg of dried solids. The solids comprised Form J.

In another preparation, 40 mg of Compound (I) (Form A) was weighted and 500 μL of THF:water (water activity of 0.85) was added. The slurries were stirred for 3 days at 50° C. A slurry sample was collected and the sample was allowed to dry for 8 hours. The resulting solids comprised a mixture of Form J and Form A.

In another preparation, 40 mg of Compound (I) (Form A) was weighted and 500 μL of THF:water (water activity of 0.95) was added. The slurries were stirred for 3 days at 5° C. A slurry sample was collected and the sample was allowed to dry for 8 hours. The resulting solids comprised a mixture of Form J and Form A.

### Example 6: Preparation of Crystalline Form K of Compound (I)

Form K of Compound (I) was prepared as follows: approximately 753.5 mg of Form F of Compound (I) were vacuum dried at 50° C. for 3 hours and exposed to 33% RH at ambient temperature for 5 days, yielding 263.3 mg of solids. The solids comprised Form K.

In another preparation, 100 mg of Compound (I) was dissolved in a mixture of 4.75 mL tetrahydrofuran and 0.25 mL water. 50 μL of this solution was dispensed into a well of a 96-well plate. Solvent was evaporated. Then THF:water 1:2 (v/v) was added into the well. The plate was sealed and exposed to the following temperature cycling procedure, which was repeated nine times: isothermal at 50° C. for 2 hours; cool to 20° C. over 8 hours in a linear fashion; heat to 50° C. The plate was then unsealed and solvent was evaporated. The solids comprised Form K.

### Synthesis of 6-(cyclopropanecarboxamido)-4-((2-methoxy-3-(1-methyl-1H-1,2,4-triazol-3-yl)phenyl) amino)-N-(methyl-d₃)pyridazine-3-carboxamide

### Step 1: Preparation of Compound 2

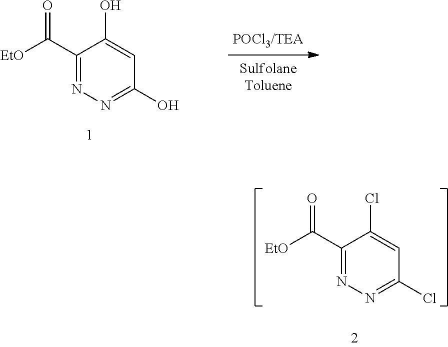

To a glass lined reactor were charged toluene (0.26 kg), sulfolane (3.4 kg), Compound 1 (1.0 kg) and POCl₃ (2.7 kg). The crude was cooled to 0° C. Triethylamine (0.89 kg) was charged, and the resulting crude mixture was heated to 65° C. and aged till the reaction reached completion. The reaction mass was cooled to 5° C.

In a separate reactor, water (7.5 kg) was charged and cooled to 5° C. The reaction mass was added slowly to the water solution, maintaining the internal temperature below 5° C. Additional water (0.5 kg) was used to rinse the reactor and aid the transfer. The resulting mixture was agitated at 5° C. for 3 hours, then extracted with MTBE three times (3×4.5 kg). The combined organic layers were washed sequentially with aqueous pH 7 buffer solution (5.0 L/kg, 15 wt % KH₂PO₄/K₂HPO₄) and water (2.5 kg). The crude was distilled under vacuum until total volume became approximately 3 L/kg. ACN (2×6.3 kg) was added followed by additional distillations back to ˜3 L/kg. The crude was cooled to 20° C. to afford Compound 2 as a 30-36 wt % solution in 90-95% yield.

### Step 2: Preparation of Compound 3

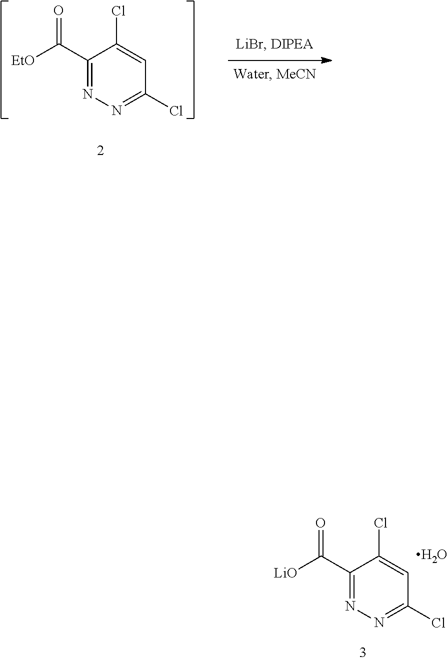

ACN (2.7 kg), lithium bromide (1.18 kg) and water (0.65 kg) were charged to a glass-lined reactor at 25° C. Compound 2 crude solution prepared above (limiting reagent) was added, followed by DIPEA (1.82 kg). The resulting slurry was agitated at 25° C. until the reaction reached completion. The product was isolated by filtration. The crude solid was washed with ACN (1.6 kg). The cake was dried under vacuum at 45° C. Compound 3 was isolated in 98 AP and 83% yield.

### Step 3: Preparation of Compound 8

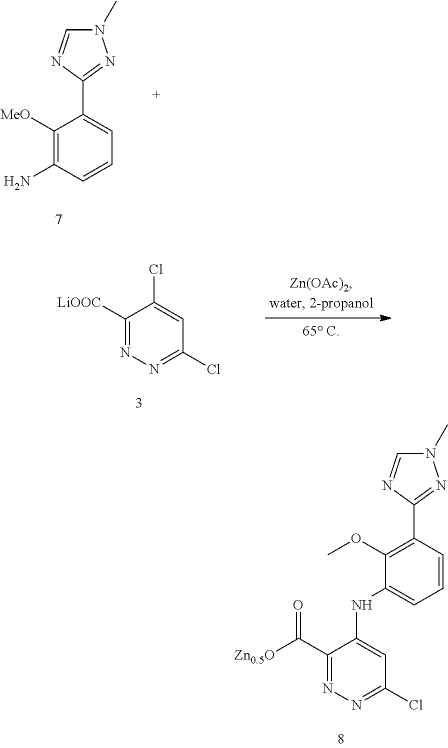

Water (6.0 kg, 6.0 L/kg) and Compound 7 (1.0 kg) were charged to a glass-lined reactor at 25° C. Zinc acetate dehydrate (1.08 kg, 1.0 equiv) was added, followed by Compound 3 (1.28 kg, 1.20 equiv). The reactor line was rinsed with 2-propanol (0.79 kg, 1.0 L/kg) and water (1.50 kg, 1.50 L/kg). The resulting homogeneous solution was heated to 65° C. and aged until the reaction reached completion. Water (7.0 kg, 7.0 L/kg) was added, and the crude mixture was cooled to 20° C. and aged for 30 min. The product was isolated by filtration. The crude solid was washed sequentially with water (6.0 kg, 6.0 L/kg), water (6.0 kg, 6.0 L/kg), THF (5.3 kg, 6.0 L/kg) and THF (5.3 kg, 6.0 L/kg). The cake was dried under vacuum at 70° C. Compound 8 was isolated in 98 AP and 94% yield.

### Step 4: Preparation of Compound 9

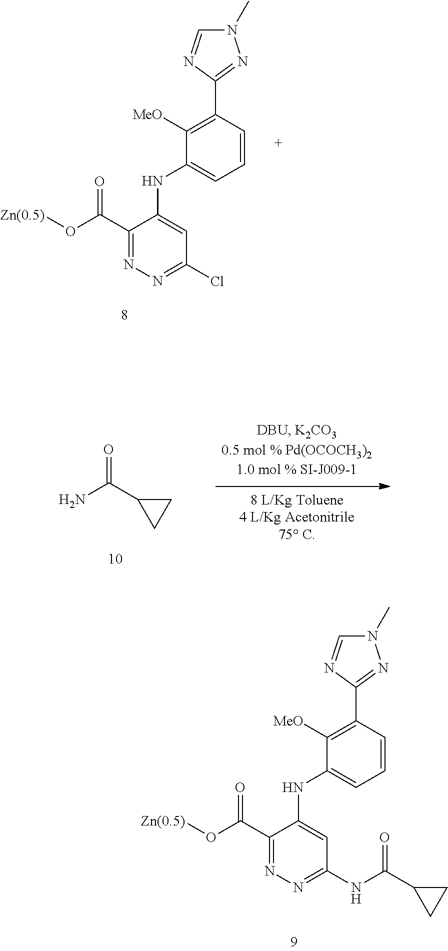

A separate glass-lined reactor was flushed with nitrogen. Toluene (0.87 kg, 1.0 L/kg) and MeCN (0.79 kg, 1.0 L/kg) were charged, followed by (2R)-1-[(1R)-1-[bis(1,1-dimethylethyl) phosphino]ethyl]-2-(dicyclohenxyphosphino)ferrocene (Josiphos SL-009-01) (14.1 g, 1.0 mol %) and palladium acetate (2.9 g, 0.5 mol %). The reactor line was rinsed with toluene (0.43 kg, 0.5 L/kg). The resulting pre-formed catalyst solution was kept under nitrogen until further usage.

At 20° C., toluene (3.46 Kg, 4.0 L/kg) and ACN (1.57 kg, 2.0 L/kg) were charged to a glass-lined reactor flushed with nitrogen. Compound 8 (1.00 kg) was added, followed by DBU (0.39 kg, 1.00 equiv). The reactor line was rinsed with toluene (0.43 kg, 0.5 L/kg). Compound 10 (0.54 kg, 2.5 equiv) and K₂CO₃ (325 mesh grade, 0.70 kg, 2.0 equiv) were added to the reaction mixture, followed by toluene (1.30 kg, 1.5 L/kg) and ACN (0.79 kg, 1.0 L/kg). The pre-formed catalyst solution was transferred into the reaction mixture, which was then heated to 75° C. and agitated until the reaction reached completion.

The reaction crude was cooled to 20° C. Aqueous acetic acid (50 Volume %, 4.0 kg, 4.0 L/kg) was charged slowly over the course of 1 h. Glacial acetic acid (10.5 kg, 10.0 L/kg) was then added. The resulting homogeneous solution was washed twice with heptane (2×3.42 kg, 2×5.0 L/kg). The bottom aqueous layer was collected and transferred to a clean reactor. Water (5.0 kg, 5.0 L/kg) was added, followed by Compound 9 seeds (0.01 kg, 1.0 wt %). The slurry was aged for 2 h at 20° C. Additional water (2.0 kg, 2.0 L/kg) was added, and the slurry was further aged for 6 h. The product was isolated by filtration. The crude cake was washed with aqueous ACN (50 Volume %, 4.5 kg, 5.0 L/kg) followed by ACN (3.9 kg, 5.0 L/kg). The cake was dried under vacuum at 65° C. Compound 9 was isolated in 98.5 AP and 84% yield.

### Step 5: Preparation of the Compound (I)

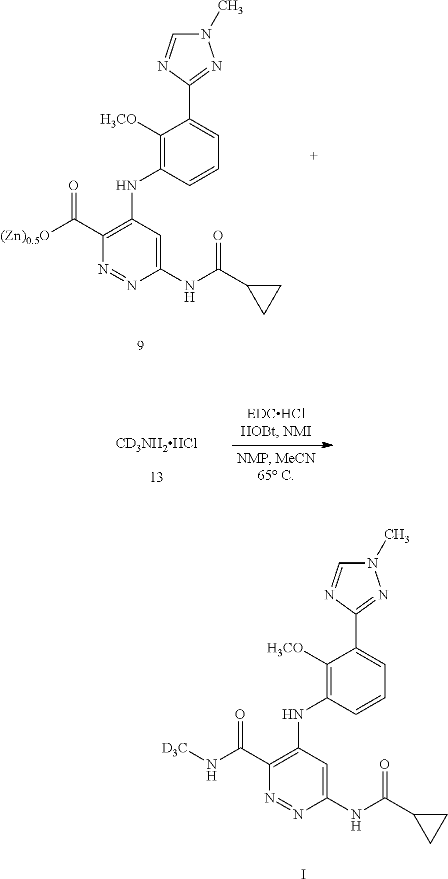

NMP (2.06 kg, 2.0 L/kg) and ACN (0.78 kg, 1.0 L/kg) were charged to a glass-lined reactor and agitated at 20° C. N-Methylimidazole (0.13 kg, 0.7 equiv.), Compound 13 (0.17 kg, 1.2 equiv.) and Compound 9 (1.00 kg) were charged to the reaction mixture. The mixture was heated to 65° C. and aged until homogeneous. HOBt 20% wet (0.17 kg, 0.5 eq), followed by EDC HCl (0.54 kg, 1.4 eq) were then charged to the reaction mixture. The reactor was rinsed with ACN (0.78 kg, 1.0 L/kg), then the resulting mixture was aged at 65° C. until the reaction reached completion. The reaction was quenched by charging water (1.0 kg, 1 L/kg), then diluted with ACN (3.0 kg, 3 L/kg). The reaction mixture was aged at 65° C. for 1 h, before cooling to 0° C., and aged for an additional 12 h at 0° C. The product was isolated by filtration. The wet cake was washed with 2:1 Water:ACN (2.8 kg, 3 L/kg) then ACN (2.4 kg, 3 L/kg), before drying under full vacuum at 65° C. Compound (I) was isolated in >99.5% purity and 91% yield.

NMP (6.2 kg, 6.0 L/kg) and Compound (I) (1.0 kg) were charged to a glass-lined reactor. The batch was heated to 70° C. to form a pale yellow solution, which was then transferred through a polish filter to a clean vessel at 70° C. 2-Propanol (2.4 kg, 3 L/kg) was added, followed by Compound I seeds (0.005 kg, 0.005 kg/kg). After aging for 1 h, additional 2-propanol (4.8 kg, 6 L/kg) was charged over the course of 2 h (3 L/kg/hr). The slurry was aged for 1 h at 70° C., cooled slowly to 0° C. and aged for additional 12 h at 0° C. Product was isolated by filtration. The wet cake was washed with 2-propanol (2×3.1 kg, 2×4 L/kg) before drying under full vacuum at 65° C. Compound (I) was isolated in >99.9% purity and 83% yield.

### Preparation of Compound 7

### Step 1: Preparation of N-methyl-N-formyl hydrazine

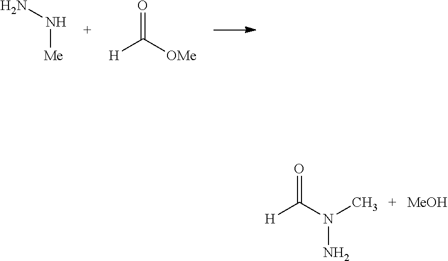

To a glass lined reactor were charged methanol (1.6 kg/kg, 2.0 L/kg) and methyl hydrazine (1 kg) at 0° C. Methyl formate (0.57 kg/kg, 1.1 equiv.) was added drop-wise. The crude was warmed up to 20° C. and aged for additional 6 h. The crude was distilled under vacuum until total volume became approximately 0.5 L/kg. Five put/take distillations with 2-MeTHF (5×3.6 kg/kg) were undertaken for the purpose of azeotropic drying. The crude was cooled to 20° C. N-Methyl-N-formyl hydrazine was isolated as 89-90 wt % solution in 89-91% yield.

### Step 2: Preparation of Compound 5

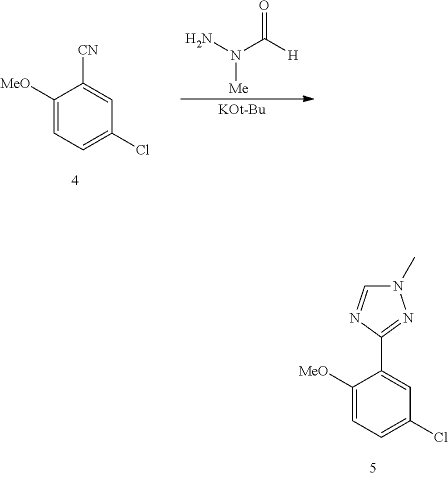

To a glass lined reactor were charged potassium tert-butoxide (1.5 kg/kg, 2.4 equiv) and THF (12.2 kg/kg) at 0° C. A mixture of Compound 4 (1.0 kg), N-methyl-N-formyl hydrazine (1.0 kg/kg, 2.30 equiv) and THF (5.3 kg/kg, 6.0 L/kg) was added slowly. The reactor line was rinsed with THF (0.5 kg/kg). The reaction crude was aged at 0° C. until reaction reached completion. Water (5.0 kg/kg) was added, and the resulting mixture was aged at 0° C. for 30 min, heated to 40° C. and aged for additional 30 min. The layers were separated and the aqueous layer was discarded. The organic layer was washed with brine (15 wt %, 5.7 kg/kg) before distilling under vacuum until total volume became approximately 5 L/kg. Four put/take distillations with ethyl acetate (4×10 L/kg) were undertaken for the purpose of azeotropic drying. The crude was cooled to 20° C. Sulfuric acid (0.66 kg/kg, 1.10 equiv.) was added, and the slurry was agitated for 2-3 h. Product was isolated by filtration. The cake was consecutively washed with ethyl acetate (2×6.5 L/kg) and heptane (8 L/kg), and dried under vacuum at 45° C. Compound 5 was isolated in 99 AP and 83% yield.

### Step 3: Preparation of Compound 6

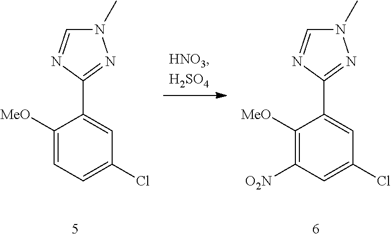 [SMILES: COc1ccc(Cl)cc1-c1ncn(C)n1 ; COc1c(-c2ncn(C)n2)cc(Cl)cc1[N+](=O)[O-]]

To a glass lined reactor were charged concentrated sulfuric acid (4.5 kg/kg) and Compound 5 (1.0 kg) at 0-5° C. Nitric acid (68 wt %, 0.35 kg/kg, 1.2 equiv) was added drop-wise. The mixture was agitated at 0-5° C. until reaction reached completion.

In a separate reactor, water (12 kg/kg) and methanol (6.5 kg/kg, 8.3 L/kg) were mixed well at 20° C. The nitration crude was transferred slowly into the methanol water mixture. The reactor line was rinsed with methanol (0.5 kg/kg). The crude was heated to 40-45° C. Aqueous ammonium hydroxide (25 wt %, 7.4 kg/kg) was added slowly. The resulting slurry was cooled to 20° C. and agitated for 3 h. Product was isolated by filtration. The cake was washed with water (2×6 L/kg), and dried under vacuum at 45° C. Compound 6 was isolated in 99 AP and 95% yield.

### Step 4: Preparation of Compound 7

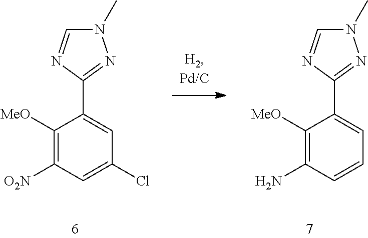 [SMILES: COc1c(-c2ncn(C)n2)cc(Cl)cc1[N+](=O)[O-] ; COc1c(N)cccc1-c1ncn(C)n1]

To a high pressure reactor flushed with nitrogen were charged methanol (8.0 kg/kg) and Compound 6 (1.0 kg). With careful exclusion of oxygen, sodium bicarbonate (0.6 kg/kg, 2.0 equiv.) and Pd/C (10% loading, 50% wet, 0.02 kg/kg) were added. The reactor was pressurized with hydrogen (41-46 psi), and the reaction mixture was aged at 20° C. for 6 h then heated to 45° C. and aged till reaction reached completion. The reactor was flushed with nitrogen, and the reaction crude was filtered to remove Pd/C. Methanol (5 kg/kg) was used to aid the transfer. The combined filtrates were distilled under vacuum until total volume became approximately 2.5 L/kg. Water (10 kg/kg) was added, and the crude was distilled under vacuum until total volume became approximately 2.5 L/kg. The crude was heated to 70° C. Brine (25 wt %, 9.0 kg/kg) was added, and the resulting crude was agitated for 6 h at 70° C. After cooling down to 0° C., the crude was further aged for 6 h. Product was isolated by filtration. The cake was washed with brine (pre-cooled to 0° C., 25 wt %, 2.0 kg/kg), and dried under vacuum at 45° C. Compound 7 was isolated in 99 AP and 88% yield.

### Preparation of Compound 13

### Step 1: Preparation of Compound 11 and Compound 12

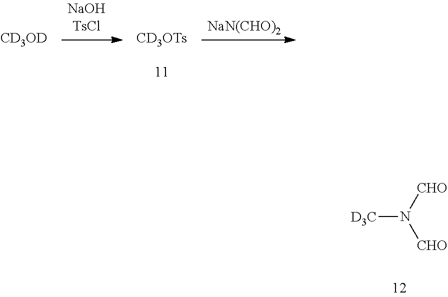

To a glass lined reactor flushed with nitrogen were charged water (16.3 L/kg) and sodium hydroxide (3.3 kg, 3.0 equiv). The mixture was aged till sodium hydroxide reached full dissolution. The crude was cooled to 0° C. d₄-Methanol (1.0 kg) and THF (4.5 L/kg) were charged. A solution of TsCI (6.3 kg, 1.2 equiv) in THF (6.3 kg, 7.1 L/kg) was added over the course of 2 h. The crude was agitated at 0° C. until reaction reached completion. The batch was warmed to 20° C. The layers were separated. The collected organic layer was diluted with MTBE (4.0 kg, 5.4 L/kg), washed with brine twice (25 wt %, 4.0 kg followed by 12 kg). The organic layer was distilled under vacuum until total volume became approximately 10 L/kg. Two put/take distillations with ACN (2×10 L/kg) were undertaken for the purpose of azeotropic drying. The crude was cooled to 20° C. ACN (10.0 kg, 12.8 L/kg) and NaN(CHO)₂ (3.3 kg, 1.2 equiv.) were added. The crude was heated to 65° C. and agitated until reaction reached completion. After cooling down to 5° C., the mixture was filtered, and the crude cake was washed with ACN twice (2×2.5 kg, 2×3.2 L/kg). The combined filtrates were distilled under vacuum until total volume became approximately 3 L/kg. The crude was cooled to 20° C. Compound 12 was isolated as an oil with 80-85 wt % in 60-70% yield.

### Step 2: Preparation of Compound 13

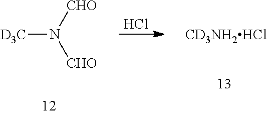

To a glass lined reactor were charged Compound 12 (1.0 kg) and methanol (3.9 kg, 5.0 L/kg) at 20° C. A solution of HCl in IPA (5-6 Normal, 4.5 kg, 1.5 equiv) was added. The resulting mixture was heated to 50° C. and agitated until reaction reached completion. THF (10 kg, 11.2 L/kg) was added slowly and the crude was cooled to 0° C. over 2 h to afford a slurry. The product was isolated by filtration. The cake was washed with THF (3.7 kg, 4.1 L/kg), and dried under vacuum at 45° C. Compound 13 was isolated in 80% yield.

### Optional Recrystallization of Compound 13

Methanol (5.6 kg, 8.3 L/kg) and Compound 13 (1.0 kg) were charged to a glass-lined reactor. DBU (0.1 kg) was added slowly. The crude was agitated for 1 h. THF (12.4 kg, 13.9 L/kg) was added slowly, and the resulting slurry was aged for 2 h. The product was isolated by filtration. The cake was washed with THF (2.6 kg, 2.9 L/kg), and dried under vacuum at 45° C. Compound 13 was isolated in 60% yield (1^{st} crop). The mother liquor was distilled under vacuum until total volume became approximately 1 L/kg. Two put/take distillations with methanol (2×2.8 kg, 2×3.6 L/kg) were performed and the solution was concentrated back to ˜1 L/kg. The crude was cooled to 20° C. THF (4.8 kg, 5.4 L/kg) was added, and the resulting slurry was aged for 2 h. The product was isolated by filtration. The cake was washed with THF (1.0 kg), and dried under vacuum at 45° C. Compound 13 was isolated in 25% yield (2^{nd} crop).

### 1. Single Crystal X-Ray Measurements

Single crystal X-ray data of Form F of Compound (I) were collected using a Bruker X8 Kappa diffractometer equipped with APEX II CCD detector and Microstar Rotating Anode X-ray source of monochromatic Cu Kα radiation.

Indexing and processing of the measured intensity data were carried out with the APEX2 program suite (Bruker AXS, Inc., 5465 East Cheryl Parkway, Madison, WI 53711 USA).

The final unit cell parameters were determined at 296 K using the full data set. The structures were solved by direct methods and refined by full-matrix least-squares approach using the SHELXTL software package (G. M. Sheldrick, SHELXTL v6.14, Bruker AXS, Madison, WI USA.). Structure refinements involved minimization of the function defined by Σw(|Fₒ|−|F_{c}|)², where w is an appropriate weighting factor based on errors in the observed intensities, Fₒ is the structure factor based on measured reflections, and F_{c} is the structure factor based on calculated reflections. Agreement between the refined crystal structure model and the experimental X-ray diffraction data was assessed by using the residual factors R=Σ∥Fₒ|−|F_{c}∥/Σ|Fₒ| and wR=[Σw(|Fₒ|−|F_{c}|)²/Σw|Fₒ|]^{1/2}. Difference Fourier maps were examined at all stages of refinement. All atoms were refined with isotropic thermal displacement parameters. Hydrogen atoms were introduced using idealized geometry with isotropic temperature factors and included in structure factor calculations with fixed parameters.

### 2. Powder X-Ray Diffraction

### PANalytical X′Pert PRO MPD Diffractometer—Transmission Geometry

X-ray powder diffraction patterns were collected with a PANalytical X′Pert PRO MPD diffractometer using an incident beam of Cu radiation produced by an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the Si 111 peak position. A specimen of the sample was sandwiched between 3 μm thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v.5.5.

### 3. Differential Scanning calorimetry

Differential scanning calorimetry (DSC) was performed using a Mettler-Toledo DSC3+ differential scanning calorimeter. A tau lag adjustment was performed with indium, tin, and zinc. The temperature and enthalpy were adjusted with octane, phenyl salicylate, indium, tin, and zinc. The adjustment was then verified with octane, phenyl salicylate, indium, tin, and zinc. The sample was placed into a hermetically sealed aluminum DSC pan, and the weight was accurately recorded. The pan lid was pierced then inserted into the DSC cell. A weighed aluminum pan configured as the sample pan was placed on the reference side of the cell. Data was collected from −20° C. to 350° C. or room temperature to 350° C. at a heating rate of 10° C./min.

### 4. Thermal Gravimetric Analysis

Thermal gravimetric analysis (TGA) was performed using a Mettler-Toledo TGA/DSC3+ analyzer. Temperature calibration was performed using calcium oxalate, indium, tin, and zinc. The sample was placed in an aluminum pan. The sample was sealed, the lid was pierced, and the sample was then inserted into the TG furnace. The furnace was heated under nitrogen with flow of 50 mg/mL. The method included heating from ambient temperature to 350° C. at a heating rate of 10° C./min.

What is claimed is:

1. Crystalline Form F of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

2. The crystalline form according to claim 1 characterized by at least one of the following:
(i) a unit cell parameters substantially equal to the following:
a=54.50±0.10 Å
b=7.83±0.10 Å
c=23.43±0.10 Å
α=90.0°
β=102.4±1.0°
γ=90.0°
Space group: C2/c
Molecules per unit cell (Z): 16,
wherein the unit cell parameters of Form F of Compound (I) are measured at a temperature of about 296 K;
(ii) a powder X-ray diffraction (PXRD) pattern comprising two or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern is measured at room temperature;
(iii) an observed powder X-ray diffraction (PXRD) pattern substantially as shown in FIG. 1, wherein the PXRD pattern is measured at room temperature.

3. The crystalline form according to claim 1 characterized by a powder X-ray diffraction (PXRD) pattern comprising three or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of the crystalline form is measured at room temperature.

4. The crystalline form according to claim 1 characterized by a powder X-ray diffraction (PXRD) pattern comprising four or more 2θ values in degrees (CuKα) selected from: 3.3±0.2, 6.6±0.2, 7.7±0.2, 9.0±0.2, 11.2±0.2, 15.0±0.2, 18.9±0.2, 20.1±0.2, 23.8±0.2, 25.3±0.2, and 27.2±0.2, wherein the PXRD pattern of the crystalline form is measured at room temperature.

5. The crystalline form according to claim 1 characterized by a powder X-ray diffraction pattern (PXRD) comprising 2θ values in degrees (CuKα) at 3.3±0.2, 7.7±0.2, and 9.0±0.2, wherein the PXRD pattern of the crystalline form is measured at room temperature.

6. The crystalline form according to claim 1 characterized by a powder X-ray diffraction pattern (PXRD) comprising 2θ values in degrees (CuKα) at 3.3±0.2, 7.7±0.2, 9.0±0.2, and 25.3±0.2, wherein the PXRD pattern of the crystalline form is measured at room temperature.

7. The crystalline form according to claim 1 wherein said Form F is in substantially pure form.

8. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form F.

9. A composition according to claim 8, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form F.

10. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form F.

11. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form F.

12. A pharmaceutical composition comprising the crystalline form according to claim 1 and a pharmaceutically acceptable carrier or diluent.

13. A pharmaceutical composition comprising crystalline Form F of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form F is characterized by a powder X-ray diffraction (PXRD) pattern comprising 2θ values in degrees (CuKα) at 3.3±0.2 and 7.7±0.2, wherein the PXRD pattern is measured at room temperature.

14. Use of the crystalline Form F according to any one of claims 1-7 in a method of preparing amorphous Compound (I).

15. Crystalline Form G of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

16. The crystalline form according to claim 15 characterized by at least one of the following:
(i) a powder X-ray diffraction (PXRD) pattern comprising two or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern is measured at room temperature;
(ii) an observed powder X-ray diffraction (PXRD) pattern substantially as shown in FIG. 2, wherein the PXRD pattern is measured at room temperature.

17. The crystalline form according to claim 15 characterized by a powder X-ray diffraction (PXRD) pattern comprising three or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of the crystalline form is measured at room temperature.

18. The crystalline form according to claim 15 characterized by a powder X-ray diffraction (PXRD) pattern comprising four or more 2θ values in degrees (CuKα) selected from: 3.2±0.2, 5.6±0.2, 8.6±0.2, 11.7±0.2, 14.2±0.2, 15.0±0.2, 17.3±0.2, and 18.2±0.2, wherein the PXRD pattern of the crystalline form is measured at room temperature.

19. The crystalline form according to claim 15 characterized by a powder X-ray diffraction pattern (PXRD) comprising 2θ values in degrees (CuKα) at 3.2±0.2 and 5.6±0.2, wherein the PXRD pattern of the crystalline form is measured at room temperature.

20. The crystalline form according to claim 15 characterized by a powder X-ray diffraction pattern (PXRD) comprising 2θ values in degrees (CuKα) at 3.2±0.2, 5.6±0.2, and 8.6±0.2, wherein the PXRD pattern of the crystalline form is measured at room temperature.

21. The crystalline form according to claim 15 wherein said Form G is in substantially pure form.

22. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form G.

23. A composition according to claim 22, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form G.

24. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form G.

25. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form G.

26. A pharmaceutical composition comprising the crystalline form according to claim 15 and a pharmaceutically acceptable carrier or diluent.

27. A pharmaceutical composition comprising crystalline Form G of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form G is characterized by a powder X-ray diffraction (PXRD) pattern comprising 2θ values in degrees (CuKα) at 3.2±0.2 and 5.6±0.2, wherein the PXRD pattern is measured at room temperature.

28. Use of the crystalline Form G according to any one of claims 15-21 in a method of preparing amorphous Compound (I).

29. Crystalline Form H of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

30. The crystalline form according to claim 29 characterized by at least one of the following:
(i) a powder X-ray diffraction (PXRD) pattern comprising two or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern is measured at room temperature;
(ii) an observed powder X-ray diffraction (PXRD) pattern substantially as shown in FIG. 3, wherein the PXRD pattern is measured at room temperature.

31. The crystalline form according to claim 29 characterized by a powder X-ray diffraction (PXRD) pattern comprising three or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of the crystalline form is measured at room temperature.

32. The crystalline form according to claim 29 characterized by a powder X-ray diffraction (PXRD) pattern comprising four or more 2θ values in degrees (CuKα) selected from: 8.2±0.2, 9.0±0.2, 13.8±0.2, 21.2±0.2, 22.4±0.2, 23.2±0.2, 24.4±0.2, 25.0±0.2, and 27.8±0.2, wherein the PXRD pattern of the crystalline form is measured at room temperature.

33. The crystalline form according to claim 29 characterized by a powder X-ray diffraction pattern (PXRD) comprising 2θ values in degrees (CuKα) at 8.2±0.2 and 21.2±0.2, wherein the PXRD pattern of the crystalline form is measured at room temperature.

34. The crystalline form according to claim 29 characterized by a powder X-ray diffraction pattern (PXRD) comprising 2θ values in degrees (CuKα) at 8.2±0.2, 13.8±0.2, and 21.2±0.2, wherein the PXRD pattern of the crystalline form is measured at room temperature.

35. The crystalline form according to claim 29 wherein said Form His in substantially pure form.

36. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form H.

37. A composition according to claim 36, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form H.

38. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form H.

39. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form H.

40. A pharmaceutical composition comprising the crystalline form according to claim 29 and a pharmaceutically acceptable carrier or diluent.

41. A pharmaceutical composition comprising crystalline Form H of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form H is characterized by a powder X-ray diffraction (PXRD) pattern comprising 2θ values in degrees (CuKα) at 8.2±0.2 and 21.2±0.2, wherein the PXRD pattern is measured at room temperature.

42. Use of the crystalline Form H according to any one of claims 29-35 in a method of preparing amorphous Compound (I).

43. Crystalline Form I of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

44. The crystalline form according to claim 43 characterized by at least one of the following:
(i) a powder X-ray diffraction (PXRD) pattern comprising two or more 2θ values in degrees (CuKα) selected from: 3.3±0.2, 5.8±0.2, 8.9±0.2, and 14.6±0.2, wherein the PXRD pattern is measured at room temperature;
(ii) an observed powder X-ray diffraction (PXRD) pattern substantially as shown in FIG. 4, wherein the PXRD pattern is measured at room temperature.

45. The crystalline form according to claim 43 characterized by a powder X-ray diffraction (PXRD) pattern comprising three or more 2θ values in degrees (CuKα) selected from: 3.3±0.2, 5.8±0.2, 8.9±0.2, and 14.6±0.2, wherein the PXRD pattern of the crystalline form is measured at room temperature.

46. The crystalline form according to claim 43 characterized by a powder X-ray diffraction (PXRD) pattern comprising 2θ values in degrees (CuKα) at 3.3±0.2 and 5.8±0.2, wherein the PXRD pattern of the crystalline form is measured at room temperature.

47. The crystalline form according to claim 43 characterized by a powder X-ray diffraction pattern (PXRD) comprising 2θ values in degrees (CuKα) at 3.3±0.2, 5.8±0.2, and 8.9±0.2, wherein the PXRD pattern of the crystalline form is measured at room temperature.

48. The crystalline form according to claim 43 characterized by a powder X-ray diffraction pattern (PXRD) comprising 2θ values in degrees (CuKα) at 3.3±0.2, 5.8±0.2, 8.9±0.2, and 14.6±0.2, wherein the PXRD pattern of the crystalline form is measured at room temperature.

49. The crystalline form according to claim 43 wherein said Form I is in substantially pure form.

50. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form I.

51. A composition according to claim 50, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form I.

52. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form I.

53. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form I.

54. A pharmaceutical composition comprising the crystalline form according to claim 43 and a pharmaceutically acceptable carrier or diluent.

55. A pharmaceutical composition comprising crystalline Form I of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form I is characterized by a 3.3±0.2 and 5.8±0.2, wherein the PXRD pattern is measured at room temperature.

56. Use of the crystalline Form I according to any one of claims 43-49 in a method of preparing amorphous Compound (I).

57. Crystalline Form J of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

58. The crystalline form according to claim 57 characterized by at least one of the following:
(i) a powder X-ray diffraction pattern comprising two or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern is measured at room temperature;
(ii) an observed powder X-ray diffraction pattern substantially as shown in FIG. 5, wherein the PXRD pattern is measured at room temperature;
(iii) an endotherm with a peak maximum in the range of from about 261° C. to about 265° C.;
(iv) a differential scanning calorimetry (DSC) thermogram substantially as shown in FIG. 6.

59. The crystalline form according to claim 57 characterized by a powder X-ray diffraction (PXRD) pattern comprising three or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of the crystalline form is measured at room temperature.

60. The crystalline form according to claim 57 characterized by a powder X-ray diffraction (PXRD) pattern comprising four or more 2θ values in degrees (CuKα) selected from: 4.0±0.2, 7.4±0.2, 8.1±0.2, 8.7±0.2, 11.4±0.2, 14.8±0.2, 16.0±0.2, 16.2±0.2, 23.2±0.2, 23.5±0.2, 24.4±0.2, and 25.8±0.2, wherein the PXRD pattern of the crystalline form is measured at room temperature.

61. The crystalline form according to claim 57 characterized by a powder X-ray diffraction (PXRD) pattern comprising 2θ values in degrees (CuKα) at 7.4±0.2 and 8.1±0.2, wherein the PXRD pattern of the crystalline form is measured at room temperature.

62. The crystalline form according to claim 57 characterized by (i) a powder X-ray diffraction (PXRD) pattern comprising 2θ values in degrees (CuKα) at 7.4±0.2 and 8.1±0.2, wherein the PXRD pattern is measured at room temperature; and (ii) an endotherm with a peak maximum in the range of from about 261° C. to about 265° C.

63. The crystalline form according to claim 57 characterized by (i) a powder X-ray diffraction (PXRD) pattern comprising 2θ values in degrees (CuKα) at 7.4±0.2, 8.1±0.2, and 16.0±0.2, wherein the PXRD pattern is measured at room temperature; and (ii) an endotherm with a peak maximum in the range of from about 261° C. to about 265° C.

64. The crystalline form according to claim 57 characterized by (i) a powder X-ray diffraction (PXRD) pattern comprising 2θ values in degrees (CuKα) at 7.4±0.2 and 8.1±0.2, wherein the PXRD pattern is measured at room temperature; and (ii) a differential scanning calorimetry (DSC) thermogram substantially as shown in FIG. 6.

65. The crystalline form according to claim 57 characterized by (i) a powder X-ray diffraction (PXRD) pattern comprising 2θ values in degrees (CuKα) at 7.4±0.2, 8.1±0.2, and 24.4±0.2, wherein the PXRD pattern is measured at room temperature; and (ii) a differential scanning calorimetry (DSC) thermogram substantially as shown in FIG. 6.

66. The crystalline form according to claim 57 characterized by a thermogravimetric analysis (TGA) thermogram comprising a weight loss of less than about 0.1% when heated from about 30° C. to about 200° C.

67. The crystalline form according to claim 57 wherein said Form J is in substantially pure form.

68. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form J.

69. A composition according to claim 68, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form J.

70. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form J.

71. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form J.

72. A pharmaceutical composition comprising the crystalline form according to claim 57 and a pharmaceutically acceptable carrier or diluent.

73. A pharmaceutical composition comprising crystalline Form J of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form J is characterized by a 7.4±0.2 and 8.1±0.2, wherein the PXRD pattern is measured at room temperature.

74. Use of the crystalline Form J according to any one of claims 57-67 in a method of preparing amorphous Compound (I).

75. Crystalline Form K of 6-(cyclopropanecarboxamido)-4-((2-methoxy-3-(1-methyl -1H-1,2,4-triazol-3-yl)phenyl) amino)-N-(methyl-d₃)pyridazine-3-carboxamide.

76. The crystalline form according to claim 75 characterized by at least one of the following:
(i) a powder X-ray diffraction (PXRD) pattern comprising two or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern is measured at room temperature;
(ii) an observed powder X-ray diffraction (PXRD) pattern substantially as shown in FIG. 8, wherein the PXRD pattern is measured at room temperature;
(iii) an endotherm with a peak maximum that is below about 130° C.;
(iv) a differential scanning calorimetry (DSC) thermogram substantially as shown in FIG. 9.

77. The crystalline form according to claim 75 characterized by a powder X-ray diffraction (PXRD) pattern comprising three or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of the crystalline form is measured at room temperature.

78. The crystalline form according to claim 75 characterized by a powder X-ray diffraction (PXRD) pattern comprising four or more 2θ values in degrees (CuKα) selected from: 3.8±0.2, 7.7±0.2, 9.4±0.2, 12.1±0.2, 12.4±0.2, 15.5±0.2, 16.1±0.2, 16.3±0.2, 18.9±0.2, 23.6±0.2, 23.8±0.2, 24.3±0.2, 25.7±0.2, and 27.7±0.2, wherein the PXRD pattern of the crystalline form is measured at room temperature.

79. The crystalline form according to claim 75 characterized by a powder X-ray diffraction (PXRD) pattern comprising 2θ values in degrees (CuKα) at 7.7±0.2 and 12.1±0.2, wherein the PXRD pattern of the crystalline form is measured at room temperature.

80. The crystalline form according to claim 75 characterized by (i) a powder X-ray diffraction (PXRD) pattern comprising 2θ values in degrees (CuKα) at 7.7±0.2 and 12.1±0.2, wherein the PXRD pattern is measured at room temperature; and (ii) an endotherm with a peak maximum in the range of from room temperature to about 125° C.

81. The crystalline form according to claim 75 characterized by (i) a powder X-ray diffraction (PXRD) pattern comprising 2θ values in degrees (CuKα) at 3.8±0.2, 7.7±0.2, and 12.1±0.2, wherein the PXRD pattern is measured at room temperature; and (ii) an endotherm with a peak maximum in the range of from room temperature to about 125° C.

82. The crystalline form according to claim 75 characterized by (i) a powder X-ray diffraction (PXRD) pattern comprising 2θ values in degrees (CuKα) at 7.7±0.2 and 12.1±0.2, wherein the PXRD pattern is measured at room temperature; and (ii) a differential scanning calorimetry (DSC) thermogram substantially as shown in FIG. 9.

83. The crystalline form according to claim 75 characterized by (i) a powder X-ray diffraction (PXRD) pattern comprising 2θ values in degrees (CuKα) at 3.8±0.2, 7.7±0.2, and 12.1±0.2, wherein the PXRD pattern is measured at room temperature; and (ii) a differential scanning calorimetry (DSC) thermogram substantially as shown in FIG. 9.

84. The crystalline form according to claim 75 characterized by a thermogravimetric analysis (TGA) thermogram comprising a weight loss of about 4% when heated from room temperature to about 125° C.

85. The crystalline form according to claim 75 wherein said Form K is in substantially pure form.

86. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein at least 90 weight % of said 6-(cyclopropanecarboxamido)-4-((2-methoxy-3-(1-methyl-1H-1,2,4-triazol-3-yl)phenyl) amino)-N-(methyl -d₃)pyridazine-3-carboxamide is in crystalline Form K.

87. A composition according to claim 86, wherein at least 95 weight % of said 6-(cyclopropanecarboxamido)-4-((2-methoxy-3-(1-methyl-1H-1,2,4-triazol-3-yl)phenyl) amino)-N-(methyl-d₃)pyridazine-3-carboxamide is in crystalline Form K.

88. A composition comprising crystalline 6-(cyclopropanecarboxamido)-4-((2-methoxy-3-(1-methyl-1H-1,2,4-triazol-3-yl)phenyl) amino)-N-(methyl-d₃)pyridazine -3-carboxamide, wherein at least 95 weight % of said crystalline 6-(cyclopropanecarboxamido)-4-((2-methoxy-3-(1-methyl-1H-1,2,4-triazol-3-yl)phenyl) amino)-N-(methyl-d₃)pyridazine-3-carboxamide is crystalline Form K.

89. A composition comprising 6-(cyclopropanecarboxamido)-4-((2-methoxy-3-(1-methyl-1H-1,2,4-triazol-3-yl)phenyl) amino)-N-(methyl-d₃)pyridazine-3-carboxamide, wherein said 6-(cyclopropanecarboxamido)-4-((2-methoxy-3-(1-methyl-1H-1,2,4-triazol-3-yl)phenyl) amino)-N-(methyl-d₃)pyridazine-3-carboxamide consists essentially of crystalline Form K.

90. A pharmaceutical composition comprising the crystalline form according to claim 75 and a pharmaceutically acceptable carrier or diluent.

91. A pharmaceutical composition comprising crystalline Form K of 6-(cyclopropanecarboxamido)-4-((2-methoxy-3-(1-methyl-1H-1,2,4-triazol-3-yl)phenyl) amino)-N-(methyl-d₃)pyridazine-3-carboxamide and a pharmaceutically acceptable carrier or diluent, wherein said crystalline Form K is characterized by a 7.7±0.2 and 12.1±0.2, wherein the PXRD pattern is measured at room temperature.

92. Use of the crystalline Form K according to any one of claims 75-85 in a method of preparing amorphous Compound (I).
